(19) United States
(12) Patent Application Publication
(10) Pub. No.: US 2025/0236824 A1
(43) Pub. Date: Jul. 24, 2025

(54) A SYSTEM FOR GENERATING AND USING CARBON DIOXIDE FOR ALGAL GROWTH UTILISING AN EFFICIENT ALGAE GROWTH SYSTEM

(71) Applicant: Paradigm Fuels Pty Ltd, Mount Pleasant, Western Australia (AU)

(72) Inventors: Peter James Clifford, Mount Pleasant, Western Australia (AU); Nathan James Clifford, Mount Pleasant, Western Australia (AU); David Joshua Hack, Mount Pleasant, Western Australia (AU)

(73) Assignee: Paradigm Fuels Pty Ltd, Mount Pleasant, Western Australia (AU)

(21) Appl. No.: 18/698,505

(22) PCT Filed: Sep. 27, 2022
(86) PCT No.: PCT/AU2022/051155

§ 371 Date: Apr. 4, 2024

(30) Foreign Application Priority Data
Oct. 4, 2021 (AU) 2021903168
Nov. 30, 2021 (AU) 2021903874
Dec. 8, 2021 (AU) 2021903965
Mar. 11, 2022 (AU) 2022900591

(51) Int. Cl. C12M 1/107; C12M 1/00

(52) U.S. Cl. CPC C12M 21/04; C12M 21/02; C12M 23/40; C12M 29/06; C12M 43/02; C12M 43/06; C12M 45/00

(57) ABSTRACT

A system for generating and using carbon dioxide comprises: (a) a closed system combustion stage for combusting a fuel in a furnace and producing an off gas containing carbon dioxide; and (b) an algae growth and oxygen generation stage for receiving off gas generated in the closed system combustion stage wherein the algae growth and oxygen generation stage through the action of algae: (i) converts carbon dioxide to a chemical product, and oxygen; and (ii) provides a gas stream to the closed system combustion stage, the gas stream comprising oxygen and carbon dioxide. The closed system combustion stage may, for example, be conducted in a power station. The system is also suitable for integration with crop cultivation, for example sugar cultivation, where carbon dioxide for algal growth may be sourced from combustion of plant material, fermentation of plant material or both.

### TECHNICAL FIELD

This invention relates to a system for generating and using carbon dioxide for algal growth, for example in a process for producing a chemical product, in particular a biofuel.

### BACKGROUND ART

The following discussion of the background art is intended to facilitate an understanding of the present invention only. The discussion is not an acknowledgement or admission that any of the material referred to is or was part of the common general knowledge as at the priority date of the application.

All references, including any patents or patent applications, cited in this specification are hereby incorporated by reference. No admission is made that any reference constitutes prior art. The discussion of the references states what their authors assert, and the Applicant reserves the right to challenge the accuracy and pertinence of the cited documents. It will be clearly understood that, although a number of prior art publications may be referred to herein, this reference does not constitute an admission that any of these documents forms part of the common general knowledge in the art, in Australia or in any other country.

Biofuels may be produced from biomass, for example as described in the following studies: Borowitzka, M. A. and Moheimani, N. R. 2013, Open pond culture systems in Algae for Biofuels and Energy, Springer, pp 133-152; Carriquiry, M. A et al., 2011, Second generation biofuels: Economics and policies, Energy Policy, 39(7), pp 4222-4234; and Raeisossadati M., 2020, Luminescent solar concentrators to increase microalgal biomass productivity, PHD Thesis, Murdoch University, WA, p 42. However, these studies have shown questionable commercial viability for biofuel production from biomass. The same is also likely to be true for production of other chemical products from biomass.

Australian Provisional Patent Applications No. 2021903168, filed 4 Oct. 2021 and No. 2021903965, filed 8 Dec. 2021 and No. 2022900591 filed 11 Mar. 2022, all by the present Applicant, and the contents of which are hereby incorporated herein by reference, describe a system and method for growing organisms, in particular algae.

### SUMMARY OF INVENTION

According to a first broad aspect of the present invention, there is provided a system for generating and using carbon dioxide, the system comprising:
(a) a closed system combustion stage for combusting a fuel in a furnace and producing an off gas containing carbon dioxide; and
(b) an algae growth and oxygen generation stage for receiving said off gas generated in the closed system combustion stage wherein said algae growth and oxygen generation stage through the action of algae:
   (i) converts said carbon dioxide to a chemical product, and oxygen; and
   (ii) provides a gas stream to the closed system combustion stage, said gas stream comprising oxygen and carbon dioxide.

The present invention also provides a method for generating and using carbon dioxide, the system comprising:
(a) combusting a fuel in a furnace of a closed system combustion stage and producing an off gas containing carbon dioxide; and
(b) receiving said off gas generated in the closed system combustion stage in an algae growth and oxygen generation stage wherein said algae growth and oxygen generation stage through the action of algae:
   (i) converts said carbon dioxide to a chemical product, and oxygen; and
   (ii) provides a gas stream to the closed system combustion stage, said gas stream comprising oxygen and carbon dioxide.

According to a further broad aspect of the present invention, there is provided a system for generating and using carbon dioxide, the system comprising:
(a) a crop processing stage for processing plant material to a sugar containing material and a fuel for combustion;
(b) a closed system combustion stage for combusting plant material, from the crop processing stage, as fuel in a furnace to produce an off gas containing carbon dioxide;
(c) a closed system fermentation stage for fermenting the sugar containing material, the sugar level of the sugar containing material being selected to produce a determined ethanol content in liquor resulting from fermentation, to generate ethanol and carbon dioxide; and
(d) a closed system algae growth and oxygen generation stage for converting carbon dioxide generated in at least one of the closed system fermentation stage and closed system combustion stage, to a chemical product and oxygen; and producing a gas mixture containing oxygen and carbon dioxide
   wherein said gas mixture containing oxygen and carbon dioxide is used in combusting said plant material.

In a still further broad aspect, the present invention provides a method for generating and using carbon dioxide, the method comprising:
(a) processing crop material in a crop processing stage to a sugar containing material and a fuel for combustion;
(b) combusting plant material, from the crop processing stage, as fuel in a furnace of a closed system combustion stage to produce an off gas containing carbon dioxide;
(c) fermenting the sugar containing material in a closed system fermentation stage, the sugar level of the sugar containing material being selected to produce a determined ethanol content in liquor resulting from fermentation, to generate ethanol and carbon dioxide; and
(d) converting, in a closed algae growth and oxygen generation stage, carbon dioxide generated in at least one of the closed system fermentation stage and closed system combustion stage to a chemical product and oxygen, and producing a gas mixture containing oxygen and carbon dioxide which is used in combusting plant material in the closed system combustion stage.

In a further broad aspect, the present invention provides a system for generating and using carbon dioxide, the system comprising:
(a) a closed system combustion stage comprising a furnace providing a furnace off gas containing carbon dioxide and components selected from the group consisting of carbon monoxide, NOx, SOx, minerals and acid gases; and
(b) a closed system algae growth and oxygen generation stage for receiving said furnace off gas, wherein said closed algae growth and oxygen generation stage contains a gas comprising a mixture of carbon dioxide and oxygen, said closed algae growth and oxygen generation stage:
   (i) consuming at least a portion of the off gas components as a nutrient;
   (ii) converting said carbon dioxide to a chemical product and oxygen;
   (iii) storing a gas mixture substantially comprising carbon dioxide and oxygen; and
   (iv) providing at least a portion of said gas mixture from (iii) to the closed system combustion stage.

The present invention further provides a method for generating and using carbon dioxide, the method comprising:
(a) providing, from a closed system combustion stage comprising a furnace, a furnace off gas comprising carbon dioxide and components selected from the group consisting of carbon monoxide, NOx, SOx, minerals and acid gases; and
(b) receiving said furnace off gas in a closed system algae growth and oxygen generation stage, wherein said closed algae growth and oxygen generation stage contains a gas containing a mixture substantially of carbon dioxide and oxygen, said closed algae growth and oxygen generation stage:
   (i) consuming at least a portion of the off gas components as a nutrient;
   (ii) converting said carbon dioxide to a chemical product, and oxygen;
   (iii) storing a portion of the gas mixture substantially comprising carbon dioxide and oxygen; and
   (iv) providing a portion of said gas mixture from (iii) to the closed system combustion stage.

The term “closed” in this specification means stages and/or components and/or sub-systems of the system wherein gases should not, and desirably cannot, escape outside of the system as described as a whole. More generally, gases are preferably directed—at least in part—from one stage to another stage in systems of the present invention, advantageously in a controlled manner.

In preferred embodiments, the gas stream may—dependent on the nature of the fuel combusted—comprise a minor proportion of a nitrogen containing gas, such as NOx. Such gases, in solution, may provide a fertiliser for algae. Either or both of the off gas and gas stream may also—dependent on the nature of the fuel combusted—contain other gases selected from the group consisting of carbon monoxide, carbonic acid, SOx, acid gases and/or water vapour as well as other components such as minerals. Nitrogen does not, due to the nature of the preferred combustion gases (which exclude air), form a major proportion of the gas stream of preferred embodiments of the present invention.

The off gas may be a furnace off gas—for example as produced by a power station combusting a carbonaceous fuel—the off gas comprising carbon dioxide and components selected from the group consisting of carbon monoxide, NOx, SOx, minerals and acid gases, wherein said algae consume one or more components of the off gas as a nutrient. For example, as described above, NOx gases in solution may act as a nitrogen fertiliser for algae. SOx and other off gas components, such as minerals, may also act as nutrients in the closed algae growth and oxygen generation stage.

The chemical product, conveniently algal lipids, is desirably processed into a biofuel, such as Biodiesel (a fatty acid methyl [or ethyl] ester), Renewable Diesel (a paraffin) and/or other paraffinic fuels such as Sustainable Aviation Fuel. A range of further chemical products may be produced using carbon dioxide as a feedstock. Whatever the desired chemical product, the algae growth and oxygen generation stage advantageously includes a system for growing waterborne algae. This system may be termed a closed algae growth and oxygen generation system as described above. The carbon dioxide is converted to biomass which then contains material, in particular algal lipids, for the manufacture of biofuel. Oxygen is generated during this process.

The system for growing algae is desirably a closed system to inhibit airborne contamination, enhance species control, provide more efficient process control and manage the containment of carbon dioxide and oxygen. As to suitable algae or microalgae, in principle, any waterborne species may be selected that requires light energy (e.g. sunlight), nutrients and carbon dioxide for growth with formation of lipids convertible to biofuel. However, high lipid content is preferred and—in some embodiments—the algal or microalgal strain may require NOx and SOx tolerance. Chlorella spp may be one suitable selection, for example Chlorella vulgaris.

The present invention provides a method for capturing an oxygen and carbon dioxide gas mixture from a liquid algae growth medium, which—when implemented in a closed algae growth and oxygen generation system, presents at least a portion of said gas mixture, to a closed system combustion stage.

In turn, high concentrations of carbon dioxide gas, produced within the closed system combustion stage, allows an efficient use of the volumes of gas to be moved, stored and utilised as feedstock to the closed algae growth and oxygen generation stage. It is also possible to direct a carbon dioxide containing stream from a furnace burning other fuels (such as coal and other fossil fuels) to the closed algae growth and oxygen generation stage. This offers an option for scrubbing such streams of carbon monoxide, carbon dioxide, NOx, SOx, mineral, acid and other components of furnace off gases using the closed algae growth and oxygen generation stage. Such an option could, in another embodiment of the invention, partially substitute or remove the requirement for the fermentation and crop processing stages.

Carbon dioxide requirements in the algae growth and oxygen generation closed system are typically driven by a required carbon dioxide uptake rate of the algae or other organisms. Carbon dioxide is, as described above, produced by both fermentation and combustion at substantial concentration, and use for organism growth represents an efficient use of this carbon dioxide. In a case where oxygen is produced in the closed algae growth and oxygen generation stage, for example by algal respiration, such oxygen—preferably at substantial concentration, for example in the range 40 to 80% by volume—is desirably directed to the closed system combustion stage. Alternatively, oxygen may be stored, vented or sold—following carbon dioxide removal if necessary.

The closed system combustion stage includes at least one furnace, desirably operable in some embodiments to burn plant material or bagasse from the crop processing stage. Preferably, gas used for combustion which contains a mixture of oxygen in major proportion and carbon dioxide in minor proportion is sourced from the closed algae growth and oxygen generation stage. Carbon dioxide in the gas mixture from the closed algae growth and oxygen generation stage is the unconsumed proportion of the CO₂ gas produced during combustion and fermentation and oxygen in the mixture is produced conveniently by growth of biomass in the closed algae growth and oxygen generation stage, described below. Air is not preferred for combustion due to the high concentration of nitrogen gas which is not useful for sustaining algal growth and which takes up carbon dioxide storage space and consumes gas transfer energy, i.e. energy required for pumping the gas. Simply put, use of air is generally inefficient for use in the systems and methods described herein. The preferred closed system combustion stage therefore reduces economically detrimental issues surrounding gas handling in particular which have arisen with use of boiler flue gases as a source of carbon dioxide for algal growth.

The furnace(s) used for combustion may operate with a feed gas of approximately 40% to 80% oxygen and carbon dioxide, sourced from the closed algae growth and oxygen generation stage, to produce a high concentration of CO₂ to the closed algae growth and oxygen generation stage. In some embodiments, the feed gas may also include a nitrogen containing gas, in particular NOx though highly desirably not air. The furnace(s) desirably accommodate high burn temperatures as may result from the rapid combustion of fuel in high concentrations of oxygen. As oxygen and nitrogen dissociate at temperatures of approximately 1300° C., in a high concentration of oxygen (including residual remaining after combustion) is likely to produce a substantial quantity of NOx gases which, in a closed system, may advantageously be directed to, and absorbed by, algae growth medium in the closed algae growth and oxygen generation stage acting as a nitrogen fertiliser or nutrient. Insoluble NOx gases, such as N₂O, and not absorbed by the algae growth medium would be directed back to the closed combustion stage together with the carbon dioxide and oxygen gas from the closed algae growth and oxygen generation stage.

The system preferably includes a carbon dioxide balancing system for balancing carbon dioxide generation in the closed system combustion stage and/or closed system fermentation stage with carbon dioxide requirements in the closed algae growth and oxygen generation stage and, in particular, for organism growth to produce biomass, in particular algal biomass. Such balance allows efficient use of the plant material from the crop processing stage while maintaining an appropriate physiological response in the algae. The carbon dioxide balancing system includes a carbon dioxide storage means.

The closed algae growth and oxygen generation stage preferably includes vessels in the form of sealed tent(s), and more preferably multi-panelled sealed tent(s) as described below, for growing waterborne algae, carbon dioxide requirements being typically driven by a required carbon dioxide uptake rate of the algae. The sealed tent(s) should be water and gas-tight during algal growth and are desirably at least partially inflatable to accommodate differing volumes of carbon dioxide, potentially at low cost. The sealed tent(s) may include—as at least part of the carbon dioxide balancing system—a carbon dioxide storage means for storing carbon dioxide generated by the closed system combustion stage and/or closed system fermentation stage in excess of the required carbon dioxide uptake rate of the algae. This would generally occur at night where for example, in the case of the fermentation process is not constrained by the lack of light and thus maintains its generation of carbon dioxide, whereas the algae growth process is constrained by the lack of light and thereby greatly reducing its carbon dioxide consumption.

As described above, sealed tent(s) are conveniently flexible with a flexible ceiling to store varying volumes of carbon dioxide. The carbon dioxide storage means of the sealed tent(s) is conveniently formed by an enclosed space between the liquid algae growth medium water level, and impervious translucent and flexible sheet(s) that seals and forms the ceiling of the sealed tent(s).

The closed system combustion stage can operate at varying burn rates by varying the amount of bagasse and/or oxygen feed, enabling flexible and intermittent operation of furnace(s). The carbon dioxide balancing system may include storage of carbon dioxide in case the closed system combustion stage and/or closed system fermentation stage generates at any one-time, an imbalance of carbon dioxide against that required to maintain the carbon dioxide uptake rate of the algae.

The joint capability to store carbon dioxide in the closed algae growth and oxygen generation stage and also control combustion rates in a closed combustion stage provides the present invention with a carbon dioxide balancing system.

The carbon dioxide balancing system may also include a means for controlling mass transfer of carbon dioxide to the carbon dioxide respiring algae. Where such algae are grown in an aqueous medium, a barrier—such as a membrane—may be placed at, or proximate to, an interface between the aqueous medium and a carbon dioxide rich gas phase to limit diffusion of carbon dioxide to the algae. A layer of bio-oil is conveniently used to form such a membrane. The material and/or thickness of the membrane may be selected to achieve the required carbon dioxide mass transfer rate through the membrane, whether formed by a bio-oil layer or otherwise. The means for controlling mass transfer of carbon dioxide to algae may also, or alternatively, include controlling the contact area of aqueous medium at an interface with a carbon dioxide rich gas phase.

A heat exchanger or heat exchanger system, for waste heat recovery, is a desirable component of the closed system combustion stage and is used to remove heat from the CO₂ exhaust gas of the furnace(s), suitable for delivery of that gas to the closed algae growth and oxygen generation stage. The heat exchanger(s) will also effectively condense water vapour (steam) and carbonic acid formed with the mixing of steam and CO₂.

A boiler is a preferable component of the closed system combustion stage and can be used to consume heat within the closed system furnace. A boiler raises boiler fluid temperature, preferably to a steam and at pressure which can be used to drive a steam turbine for the production of electricity. The steam may also be reticulated to feed a heat exchanger, to provide an ability—through provision of heat—to:
a. distill ethanol beer to an ethanol rich solution; and/or
b. to evaporate juice to molasses; and/or
c. to preheat ethanol for ethanol vapour permeation process; and/or
d. to preheat lipids for hydrogen processing; and/or
e. to preheat ethanol for dehydration into ethylene; and/or
f. to preheat ethylene for downstream processing into polyethylene and/or sustainable aviation fuel.

Boiler fluid, prior to it entering the boiler and conversion to steam, can be used as a coolant to establish a multiple stage CO₂ exhaust gas cooling method suitable to lower gas temperature of the furnace(s), for delivery of that gas to the closed algae growth and oxygen generation stage at a temperature favourable to algal growth. This method commonly utilises an “economizer” as termed in the boiler industry.

### Crop Processing Stage and Sweet Sorghum

Sugar containing material is conveniently derived from a plant or blend of plants having a relatively high sugar content and plant fibre yield. Each selected plant is desirably grown as a crop, desirably at scale, the scale conveniently being determined as a function of chemical product, for example biofuel production. Sugar cane and sweet sorghum are preferred examples of plants suitable for such crops. From this perspective, sweet sorghum is advantageous since it can yield 2 to 3 crops or tranches a year, or even some locations up to six immature crops (but rich in sugar and with sufficient plant fibre) per year, depending on water availability, compared to 1 crop or tranche per year for sugar cane. High carbon dioxide plant uptake is also desirable as this provides a carbon sequestration system of assistance in offsetting carbon emissions elsewhere.

The crop processing stage may process sugar cane or sweet sorghum—or other selected sugar imbued plant—to extract a sugar containing juice, for example by crushing, allowing the juice to be directed to the closed system fermentation stage and producing bagasse for combustion in the combustion stage. A “proximate crop processing stage” is preferred to avoid the need for a centralised milling facility and disadvantages that may arise from transportation of the crop including transport costs, time efficiencies, juice loss, plant dehydration and crystallisation and degradation of sugars. A proximate crop processing stage, allows crushing of a crop in close proximity to the crop's location thus providing transport efficiencies since transport of the crop to a regional centralised mill crushing facility and related cost is avoided. A proximate crop processing stage conveniently comprises at least a crushing unit. Desirably, the proximate crop processing stage also includes a refining unit to concentrate the juice obtained by crushing by evaporation, or alternatively membrane processes (for example ultrafiltration then reverse osmosis), optionally to molasses. Production of molasses advantageously reduces storage requirements for sugar containing juice, facilitating year-round operation of the closed system fermentation stage. Bagasse may be similarly stockpiled.

### CO₂ Feed to the Algae Growth and Oxygen Generation System

Without wishing to be bound by theory, if a molecular balance of lipid and carbohydrate component creation is considered, a process which occurs in algae growth within the closed algae growth and oxygen generation stage, there is a very approximate equivalent amount of 138 moles of oxygen released by the algae to 114 moles of CO₂ consumed by the algae (which when converted to weight is very approximately equal).

The combustion of cellulose and lignins (or alternative fuels such as coal) in furnace(s) of the combustion stage generates very approximately 1 mole of CO₂ to every mole of oxygen consumed. Thus, on balance, the generation of oxygen in the closed algae growth and oxygen generation stage can exceed the required consumption of oxygen in the furnace(s) of the combustion stage, which in turn sustains the CO₂ delivered for algae growth in the closed algae growth and oxygen generation stage.

In a balanced system where the closed algae growth and oxygen generation stage is dimensioned to consume all CO₂ produced by the furnace, there is not as much oxygen required to produce CO₂ in the closed combustion stage as oxygen produced by the closed algae growth and oxygen generation stage and there is likely to be an oxygen surplus. This oxygen gas will ultimately need to be vented or used in downstream production of resale gases. Added to this imbalance, is the CO₂ generated in the closed system fermentation stage, which has no requirement for free oxygen and this CO₂ can also be delivered to the closed algae growth and oxygen generation system.

Oxygen is therefore expected to be a byproduct of the system, which—if commercialised—must be cleansed of CO₂ and minor component gases before marketing as oxygen.

On start-up, the system may be primed by operating the closed system fermentation stage to produce carbon dioxide for the algae growth and oxygen generation stage without operating the combustion stage or not using carbon dioxide from combustion to support the algae growth and oxygen generation stage. At least a seeding quantity of algae is introduced to the system on start up as well.

### Production of Ethanol from Fermentation

The closed system fermentation stage is conveniently conventional in design and operation. The product ethanol stream, which may also be referred to as a primary ethanol stream, from the closed system fermentation stage has a relatively low ethanol content when considered from a perspective of use as a commercial product. Preferably, the system includes an ethanol concentration sub-system to increase the ethanol content to an acceptable level for use as fuel or for other purposes. The ethanol concentration sub-system desirably includes a distillation stage to capture a high ethanol content distillate ethanol stream, preferably through multi-stage distillation.

The ethanol distillate may then be further purified using a membrane processing stage, such as a vapour permeation stage.

Ethanol can then be supplied as a commercial product, or dehydrated to produce ethylene. Ethylene production from ethanol by dehydration is conveniently conventional in design and the resulting ethylene gas can be further polymerized to produce polyethylene and variants of short carbon chain paraffins such as kerosene and aviation fuel.

### The Algae Growth and Oxygen Generation System

The system for growing carbon dioxide respiring organisms, forming one part of the closed algae growth and oxygen generation stage, desirably comprises at least one but more preferably a plurality of sealed tent(s), desirably “multi-panelled sealed tent(s)” as described herein, communicating with the closed system combustion stage(s) and/or closed system fermentation stage for delivery of carbon dioxide to the at least one multi-panelled sealed tent(s). In preferred embodiments, multi-panelled sealed tent(s) desirably include a combination or all of the following features:
a. isolate(s) the growing organisms from airborne pollutants and contamination by other species of organisms including parasitic organisms; which
b. enhance(s) species control;
c. assist(s) in process control; especially if (and not limiting the invention) large numbers of, for example, in excess of multiple hundreds, multi-panelled sealed tents are utilised in one installation; and
d. has/have a high utilisation efficiency of supplied CO₂ in that the CO₂ can be trapped and transferred into the liquid algae growth medium, thereby promoting algae growth; and
e. allow(s) for commercially viable high concentrations of CO₂ in the liquid algae growth medium; and
f. provide(s) variable volume storage of excess CO₂; and
g. allows nitrogen gases such as NOₓ and the optional use of NH₃ for fertiliser (which may be obtained through a solar distillation process as described below) to be contained in the multi-panelled sealed tent(s) and
h. allow(s) oxygen to be trapped and utilised both:
   i. within the process (e.g. for combustion), and
   ii. as a commercial product; and
i. allow(s) light irradiation to be simply managed and distributed, thereby promoting algae growth; and
j. provide(s) a physical structure to eliminate harmful UV, which is detrimental to algae growth.
   It will be understood that alternative vessels to multi-panelled sealed tents would also desirably include a combination or all of features (a)-(j) above.

Carbon dioxide may be captured from the closed system combustion stage and/or the closed system fermentation stage, more particularly from the furnace(s) in the closed system combustion stage and/or from still(s) comprising the closed system fermentation stage, and controllably transferred to the multi-panelled sealed tent(s) through, for example, a pressure differential system that pumps carbon dioxide from the closed system combustion stage and/or the closed system fermentation stage.

Preferably, the or each multi-panelled sealed tent of the algae growth and oxygen generation stage comprises any combination, desirably all, of the following elements:
a. a liquid algae growth medium bearing photosynthetic organisms; and
b. a light diffusion subsystem to expose organisms at depth to light energy; and
c. a cleansing system for the light diffusion subsystem to remove algae build-up that will inevitably occur on the light diffusion surfaces; and
d. end plates at each end of the multi-panelled sealed tent(s), where one end plate(s) (the “far end”) is used to extract liquid algae growth medium bearing organisms which traverse from one end (herein the “near end”) of the “multi-panelled sealed tent(s)” to the other end plate(s) (herein the “far end”); and
e. a supply of seed algae, conveniently through a “near end” plate(s), intended to develop to harvestable concentrations by the time the algae has traversed the length of the multi-panelled sealed tent(s), conveniently to the “far end” plate(s); and
f. means for the transfer and/or circulation of water through the multi-panelled sealed tent(s), as facilitated by external pumps of the multi-panelled sealed tent(s), provides a progression of algae density from seed density at one end to a higher harvestable density at the other end; and
g. a means for circulation of gases through the multi-panelled sealed tent(s) to provide an extended and elongated route for gases to travel over the algae medium for effective uptake and delivery of gas components such as carbon dioxide and NOx, whether through diffusion into the algae growth medium or, in the case of oxygen, diffusion out of the algae growth medium; and
h. a supply of nutrient, and additional water (as for example, algae growth consumes water) which can be added to the transfer and/or circulated water that provides the algae seed density; and
i. construction with flexible impervious translucent material, conveniently in the form of sheet(s), to form the multi-panelled sealed tent(s) and contain the CO₂ stored above the liquid algae growth medium; and
j. with flexible translucent sheets, as above described, which conduct sunlight and preferably contain a combination of additives selected from the group consisting of UV protection additives, stabilisers, antioxidants, brightness additives (also called luminescent additives that shift UV to violet and blue spectrum) and dye (preferably pink dye (i.e. Blueish red) to absorb some of the unwanted light frequencies to provide optimum light spectrum for the growing cycle of algae); and
k. a source of carbon dioxide (CO₂) to be delivered—desirably through, or close to, one end plate—and intended to occupy the CO₂ storage space above the liquid algae growth medium with Henry's Law and the high solubility of CO₂ in water ensuring the mass transfer of CO₂ from the CO₂ storage space by diffusion into the liquid algae growth medium enriching it with CO₂, to cultivate the algae; and
l. a gas offtake, in or near the other end plate(s) (that is, the other end of the multi-panelled sealed tent(s) from which the CO₂ is delivered) from which oxygen/carbon dioxide gas mixture as generated by the algae, can be bled from the CO₂ storage space of the or each multi-panelled sealed tent to return that gas mixture to the closed system combustion stage or be vented; and
m. external to the or each multi-panelled sealed tent, pump(s) and algae separation system(s) fitted to maintain a circulation of liquid algae growth medium (with seed organisms) through “multi-panelled sealed tent(s)”, desirably from the “far end” plate back to the “near end” plate; and
n. matter or nutrients promoting organism growth to be provided to organisms borne by the liquid algae growth medium, conveniently as supplied through the “near end” plate; and
o. optionally, a layer of ballast, such as steam sterilised soil, on the floor of the “multi-panelled sealed tent” to provide ballast and stability in times of high wind, and provide thermal ballast to assist in maintaining a more constant water temperature.

The multi-panelled sealed tent(s) is/are closed systems being managed using sensors and control systems (for example, a SCADA control system) to monitor and manage preferably, but not limited to, a combination of any, or all, of the following:
a. depth of the water;
b. the temperature of the liquid algae growth medium;
c. the pressure of gas in the “multi-panelled sealed tent”;
d. pH level of the liquid algae growth medium;
e. valves to manage the CO₂ flow into the multi-panelled sealed tent(s);
f. the O₂/CO₂ gas mixture extraction from the multi-panelled sealed tent(s);
g. the top-up water/nutrient supply;
h. the Light Diffusion Subsystem (herein “LD Device”) cleansing means.

The multi-panelled sealed tent(s) are conveniently supported along its sides by curbing. The curbing itself is preferably supported by earthworks.

The number of multi-panelled sealed tents that may be deployed in the system is selected dependent on factors such as the amount of crop crushed and bagasse produced (and hence CO₂ generated in the “closed system combustion” stage).

In preferred embodiments, each multi-panelled sealed tent contains at least one, more preferably a plurality of, Light Diffusion Device(s) (herein “LD Device(s)”) designed to provide sufficient light at depth in the multi-panelled sealed tent(s) such that the organisms borne by the liquid algae growth medium are exposed to light energy, desirably from surface to the depth of the LD Device. The LD Devices are conveniently sealed containers that may contain water which is kept separate from the liquid algae growth medium, with water in translucent sealed containers preferably containing additive(s) to reflect and/or transmit light through the sides of the LD Device.

### Preferred Configuration of LD Devices

The LD Devices of preferred embodiments of the invention are preferably aligned orthogonal to the flow of liquid algae growth medium, where each alternate LD Device (herein “Braced Row”) is secured by bracing to the floor of each multi-panelled sealed tent and allows the liquid algae growth medium to flow over the Braced Row(s). Strapped to the Braced Rows are “In-filler” LD Device containers that may conveniently be supported vertically with sufficient buoyancy induced by a gas pocket between the LD Device ceiling and its contained water level to hold the LD Device(s) upright when the base of the LD Device(s) is secured to the floor of each multi-panelled sealed tent or preferably to the Braced Rows. The “In-filler” LD Device(s) are desirably purposefully floated a sufficient distance from the floor, to allow the movement of water underneath the “In-filler” LD Device thereby sequentially directing the flow of liquid algae growth medium over the “Braced Rows”, vertically down the side of the LD Devices, to flow beneath the “In-filler” LD Device Containers and then vertically up the side of the LD Devices to then flow over the next Braced Row.

This water movement provides a more consistent light energy supply to the algae.

The flow of liquid algae growth medium in a sequence of over and then under LD Devices, as described above, encourages a single directional flow of medium from the near end of a multi-panelled sealed tent to the far end of the mulit-panelled sealed tent and avoids problems of reverse dilution with other arrangements of the LD devices.

Furthermore, that the flow of medium is directed across the braced rows at or near the surface, encourages the transfer of CO₂ and oxygen between liquid algae growth medium and the gas storage space above the liquid algae growth medium.

### Alternative Configuration of LD Devices

An alternative configuration of LD Devices, which may be used interoperably, with the Braced Row LD arrangement described above, aligns the LD Devices parallel to the flow of liquid algae growth medium. This provides a manufacturing advantage, whereby all LD Devices are manufactured to the same length, being approximately the panel width of each multi-panelled sealed tent. This alternate inventive configuration lacks the flow dynamics that are obtained when the LD Devices are configured according to the Braced Row arrangement and where the flow of liquid algae growth medium is orthogonal to (across) the LD Devices.

### Light Distribution through the LD Devices

The width of the LD Device at the surface of the liquid algae growth medium bearing organisms, is desirably of sufficient width to capture enough light that the average Photosynthetically Active Radiation commonly known as “PAR” light intensity emitted through the sides of the LD Device is at a sufficient light intensity on average, suitable for growing algae. The spacing between each LD Device is commensurate with variable algae density along the multi-panelled sealed tent (or alternative vessel) as defined by a determined profile of algae density, resulting from a seed concentration at the “near end” of the multi-panelled sealed tent (or one end of an alternative vessel) and a harvestable concentration at the “far end” (or the other end of an alternative vessel) of the multi-panelled sealed tent. The spacing, which also forms a growth passage, between each LD Device is preferably determined by the maximum light pathway distance from the side of the LD Device into the liquid algae growth medium for the determined algae density at that point along the multi-panelled sealed tent (or other vessel). Algae density may be controlled, at least in part, by movement or circulation of growth medium through the multi-panelled sealed tent (or other vessel) with algae density conveniently being measured at sampling points.

It is advantageous that the LD Devices are in close proximity where there is a high algal density (conveniently at a “far end” of a multi-panelled sealed tent) and reducing in their proximity by increasing the separation distance between the LD Devices as the concentration of algae reduces progressively to that of the seed concentration at the feed end (“near end”) of a multi-panelled sealed tent.

LD devices are conveniently arranged in parallel as a community contained within each panel of a multi-panelled sealed tent, each community of LD Devices having the same separation distance between the LD devices commensurate with the average algal density of that panel. Preferably, each panel contains the same volume of liquid algae growth medium. In such a preferred arrangement, there is varied panel length resulting from the different number of LD devices assigned to each panel. Within each panel there is a liquid volume through which liquid algae growth medium is circulated and a gas space through which a mixture of carbon dioxide/oxygen gas is circulated. A barrier is desirably provided to enable the gas space of one panel to be closed off from adjacent panels. This serves two purposes: one purpose is to force the circulation of gases along each panel (along an elongated gas flow path) to extend the gas flow route within the multi-panelled sealed tent for efficient transfer of gas components between the algae growth medium and the circulating gas. The second purpose is to, for example, mitigate against a leak in the multi-panelled sealed tent at the location of the panel. Gas flow between adjacent separated panels is controllable through the use of suitable piped/gas reticulation between panels and with suitable valve arrangements.

The LD devices are subject to algal buildup and are conveniently cleansed of algae buildup by a cleansing means preferably comprising: a combination of flexible blades that are configured for bi-directional motion. Conveniently, the cleansing means, which may conveniently straddle an LD device preferably in the form of a sealed sac, comprises at least two flexible blades, each one effective for at least one direction. Each flexible blade is preferably beveled (slanted) against the side of the LD Device with, where the flexible blade is moved across the sealed sac surface, the force of growth medium holding the blade against the side of the LD Device. Flexible blade(s) are conveniently pulled across the surface of an LD device, for example by a rope or chord.

### Optional Solar Distillation System to Concentrate Algae and Separate Lipids

An optional solar distillation system, can be inserted in the flow of liquid algae growth medium prior to the concentration of the algae medium, conveniently by centrifuge(s), for downstream biofuel processing. By using distillation trays that contain approximately 4 cm depth of medium exposed to sunlight over the length of the distillation tray and not wishing to be bound by theory, calculations show that for a high sunlight radiation region, a collection of distillation trays similar in the sum of area to that of a multi-panelled sealed tent is sufficient to boil-off approximately 50% of the daily water throughput of the medium, and at the same time lyse the algae cell (through the high temperatures and boiling action) to separate lipids for biofuel processing.

End plates may be provided at each end of the distillation tray, and preferably constructed to supply a medium waterfall at one end, and the medium supply at the other end. The distillation trays can optionally be enclosed with a transparent (to the sun) cover to trap water and possibly ammonia vapour for recycling into the same or other system stages. This optional distillation system is intended to reduce the amount of electrical energy required to drive the preferred centrifuges that concentrate the algae medium, thereby reducing internal system energy requirements for the production of sufficient electrical energy, and thereby maximizing the available biofuel for market from the system.

### Algae Harvesting and Downstream Processing

Where algae are used as carbon dioxide respiring organisms, in the algae growth and oxygen generation stage, the system includes an algae separation system to separate algae from water, from an outlet end or recirculating stream, desirably when the algae form a sufficient population density in the water at the outlet end of the growth vessel(s), say in the range 0.1 vol % to 3.0 vol % algae. The algae separation system desirably includes an optional distillation system (described in paragraph [0057] above) and a concentrator, preferably a centrifuge, which concentrates the algae into a conglomerate, for example to in the range of 40 to 60 wt % algae.

Following the algae separation system, the system preferably includes a biofuel production stage. In one embodiment, the biofuel production stage includes a device, preferably an ultrasound device, to lyse cell walls of the algae and form biomass; a separator, such as a centrifuge, to separate the biomass; and a treatment system to convert the biomass to biofuel.

A convenient treatment system to convert the biomass to biofuel, for example biodiesel or paraffinic oil, extracts lipids from the biomass to convert to biofuel.

Extraction of plant lipids from biomass conveniently involves solvent extraction with a solvent selective for the plant lipids, for example a hydrocarbon such as hexane which may be in admixture with an alcohol, conveniently ethanol. A plant lipid rich concentrate from solvent extraction is then converted to biofuel. The solvent is recovered from the plant lipids, prior to conversion to biofuel, by any suitable process, conveniently distillation.

Optional conversion of the lipids to biodiesel is conveniently achieved by transesterification with an alcohol, for example ethanol or methanol. Ethanol is preferred as forming an ethyl ester based biodiesel, not only because of the availability of ethanol within the system, but is expected to result in lower emissions, higher heat content and cetane number than a methyl ester based biofuel.

Conversion of the lipids to a paraffin oil such as Renewable Diesel or Aviation Fuel is conveniently conventional in design and operation utilizing hydrogen under high temperature and pressure over a catalyst to “hydrogen process” the lipids into paraffins using two common process stages called “Hydrotreating” and “Hydrocracking”.

The systems described above are advantageously modular. In this way, one or more of the crop processing, combustion, fermentation, algae growth and oxygen generation and Algae Harvesting and downstream processing stages and associated equipment may be provided as discrete modules which may be replaced with new modules if required to vary capacity, adopt improved technology and/or for maintenance purposes.

### Crushing to Prepare Biomass for System Processes

Use of juice extracted from plant material alone is unlikely to yield a sufficient sugar content, as represented by Brix or other scale, to generate high levels of ethanol in an ethanol product stream from the closed system fermentation stage.

In this regard, a target ethanol content is advantageously selected as high as possible, upwards of 10 vol %, without affecting conduct of the fermentation process. In this regard, fermentation is typically conducted using a microorganism—for example a yeast such as S cerevisiae spp though some bacteria may be suitable. Ethanol above a certain content, for example about 16% by volume of the fermentation liquor, is toxic to the microorganisms and adversely affects fermentation. As a counter, insufficient ethanol content increases energy requirements for distillation and may make distillation less commercially viable. This impracticality may be avoided by boosting sugar content in a feed to the closed system fermentation stage to a level that, allowing for efficient fermentation, may allow the fermentation liquor to reach the toxic ethanol content but not exceed it.

A convenient way to boost sugar content is to mix sugar containing juice with a material having an enriched sugar content. Juice may be concentrated, optionally to produce molasses, by heating and then evaporation and/or ultrafiltration then reverse osmosis. Concentration including molasses production is also preferably conducted using refining unit(s) included within the “proximate crop processing plant”. Where the juice is concentrated, the sugar containing juice may be concentrated to a sugar content (e.g 23.5 Brix (Bx) where a target ethanol content of 12 vol % ethanol is required) that allows the target ethanol content in the product ethanol stream to be reached. In this case, the sugar containing juice may be required to be used within a certain time period to avoid potential problems with product aging and decaying. Whereafter, concentrated juice (in the form of molasses) can be used to sustain the “closed system fermentation” stage once the sugar containing juice is consumed.

Desirably, the closed system combustion stage and/or closed system fermentation stage operate(s) continuously, preferably year-round, to provide carbon dioxide to the closed algae growth and oxygen generation stage. A consistent or constant feed of carbon dioxide from the closed system combustion stage and/or closed system fermentation stage to the closed algae growth and oxygen generation stage is likely to be required if organism growth is to be maintained at a rate matched with the required production rate of the chemical product, for example a biofuel. In that regard, consistent or constant input of a suitable sugar containing feedstock to the closed system fermentation stage should be maintained. The generation of CO₂ within the closed system combustion stage can be controlled to meet demand by controlling the supply of suitable plant material (bagasse) and/or oxygen to the “closed system combustion stage. Burn rate may also be controlled with a target set for carbon dioxide production. The fermentation stage and/or combustion stage desirably operate even outside a determined harvesting schedule for the crop processed in the crop processing stage.

Conveniently, solid plant material produced in the “proximate crop processing plant”, such as bagasse, is used as a fuel in the closed system combustion stage, for principally producing CO₂ for the closed algae growth and oxygen generation stage; and additionally able to heat the primary ethanol stream for distillation; and then also optionally for concentration, including production of molasses, and for boosting the sugar content of the juice directed to the closed system fermentation stage. Other uses of the solid plant material as a fuel or for other purposes, such as fertiliser or mulch, are not precluded. In this case, a closed system combustion stage typically involves furnace(s) selected from the group consisting of chain grate, fluidised bed and/or blast sealed combustion furnaces.

The system and method enables crop farming, at advantageously large scale, to be coupled with chemical production, in particular ethanol, biodiesel and/or paraffinic oil production, in an energy efficient and otherwise sustainable manner. Raw materials are used either as feedstocks or fuel within the system to maintain energy efficiency and minimise requirements for external energy inputs. Waste is kept to a minimum and little supporting infrastructure, such as roads, port facilities and utilities such as power-is required.

### BRIEF DESCRIPTION OF THE DRAWINGS

Further features of the present invention are more fully described in the following description of several non-limiting embodiments thereof. This description is included solely for the purposes of exemplifying the present invention. It should not be understood as a restriction on the broad summary, disclosure or description of the invention as set out above. The description will be made with reference to the accompanying drawings in which:

FIG. 1 is a block diagram schematically illustrating one embodiment of a system for generating and using carbon dioxide according to the present invention.

FIG. 2 is a block diagram schematically illustrating the “Proximate Crop Processing Plant” stage forming part of the system shown in FIG. 1.

FIG. 3 depicts a schematic cross section of a panel within a multi-panelled sealed tent which may be used in the closed algae growth and oxygen generation system in accordance with embodiments of the present invention.

FIG. 4 depicts a schematic long section diagram of a multi-panelled sealed tent within a closed algae growth and oxygen generation system and a relationship to other components of the invention and which may be used in accordance with embodiments of the present invention.

FIG. 5 depicts a schematic cross section of a supporting curb for each side of a multi-panelled sealed tent as may be used in the closed algae growth and oxygen generation system and which may be used in accordance with embodiments of the present invention.

FIG. 5A depicts the curbing along the lengthwise side of the multi-panelled sealed tent(s) whereas FIG. 5B includes the end plate gutter used in the curbing at the near and far ends of the multi-panelled sealed tent(s).

FIG. 6 depicts a schematic cross section of a number of Light Diffusion Devices of the closed algae growth and oxygen generation system and which may be used in accordance with embodiments of the present invention.

FIG. 7 depicts the relationship between LD Device containers secured to the floor of the multi-panelled sealed tent and the adjacent “In-filler” LD Device floated containers.

FIG. 8 depicts a schematic lay out of light diffusion devices in a multi-panelled sealed tent and which may be used in accordance with embodiments of the present invention.

FIG. 9 depicts a schematic plan and side elevation view of a Light Diffusion Device Cleansing System which may be used in accordance with embodiments of the present invention.

FIG. 10 depicts a schematic diagram of a “far end” plate of a multi-panelled sealed tent and which may be used in accordance with embodiments of the present invention.

FIG. 11 depicts a schematic diagram of a “near end” plate of a multi-panelled sealed tent and which may be used in accordance with embodiments of the present invention.

FIG. 12 depicts a schematic diagram of a winching and rope/chord system for cleansing means for light diffusion (LD) devices which may be used in accordance with embodiments of the present invention.

FIG. 13 depicts a schematic diagram by way of example of one embodiment of the present invention of approximated gas flow resulting from an example sorghum farm of an embodiment of the invention.

FIG. 14 depicts a rendered aerial schematic view of a multi-panelled sealed tent of embodiments of the present invention.

FIG. 15 depicts a schematic diagram by way of example of one embodiment of the present invention of reticulated gas flow within a multi-panelled sealed tent of embodiments of the invention.

FIG. 16 is a block diagram schematically illustrating one embodiment of an algae harvesting and downstream processing stage according to embodiments of the present invention.

FIG. 17 is a first schematic diagram showing an arrangement of distillation trays including an optional solar distillation system upstream of the algae harvesting and downstream processing stage of FIG. 16.

FIG. 18 is a second schematic diagram showing an arrangement of distillation trays in embodiments including an optional solar distillation system upstream of the algae harvesting and downstream processing stage of FIGS. 16 and 17.

FIG. 19 is a schematic diagram expounding on FIG. 1 and showing an exemplary arrangement of processes in a system according to embodiments of the present invention.

### DEFINITIONS

The following definitions are provided as general definitions and should in no way limit the scope of the present invention to those terms alone but are put forth for a better understanding of the following description.

Unless defined otherwise, all technical and scientific terms used herein have the same meaning as commonly understood by those of ordinary skill in the art to which the invention belongs. It will be further understood that terms used herein should be interpreted as having a meaning that is consistent with their meaning in the context of this specification and the relevant art and will not be interpreted in an idealized or overly formal sense unless expressly so defined herein. For the purposes of the present invention, additional terms are defined below. Furthermore, all definitions, as defined and used herein, should be understood to control over dictionary definitions, definitions in documents incorporated by reference, and/or ordinary meanings of the defined terms unless there is doubt as to the meaning of a particular term, in which case the common dictionary definition and/or common usage of the term will prevail.

For the purposes of the present invention, the following terms are defined below.

The articles “a” and “an” are used herein to refer to one or to more than one (i.e. to at least one) of the grammatical object of the article. By way of example, “an element” refers to one element or more than one element.

The term “about” is used herein to refer to quantities that vary by as much as 30%, preferably by as much as 20%, and more preferably by as much as 10% to a reference quantity. The use of the word ‘about’ to qualify a number is merely an express indication that the number is not to be construed as a precise value.

Throughout this specification, unless the context requires otherwise, the words “comprise”, “comprises” and “comprising” will be understood to imply the inclusion of a stated step or element or group of steps or elements but not the exclusion of any other step or element or group of steps or elements.

Any one of the terms: “including” or “which includes” or “that includes” as used herein is also an open term that also means including at least the elements/features that follow the term, but not excluding others. Thus, “including” is synonymous with and means “comprising”.

In the claims, as well as in the summary above and the description below, all transitional phrases such as “comprising,” “including,” “carrying,” “having,” “containing,” “involving,” “holding,” “composed of,” and the like are to be understood to be open-ended, i.e., to mean “including but not limited to”. Only the transitional phrases “consisting of” and “consisting essentially of” alone shall be closed or semi-closed transitional phrases, respectively.

Although any methods and materials similar or equivalent to those described herein can be used in the practice or testing of the present invention, preferred methods and materials are described. It will be appreciated that the methods, apparatus and systems described herein may be implemented in a variety of ways and for a variety of purposes. The description here is by way of example only.

As used herein, the term “exemplary” is used in the sense of providing examples, as opposed to indicating quality. That is, an “exemplary embodiment” is an embodiment provided as an example, as opposed to necessarily being an embodiment of exemplary quality for example serving as a desirable model or representing the best of its kind.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

The phrase “and/or”, as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one”, in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

For the purpose of this specification, where method steps are described in sequence, the sequence does not necessarily mean that the steps are to be carried out in chronological order in that sequence, unless there is no other logical manner of interpreting the sequence.

### DESCRIPTION OF PREFERRED EMBODIMENTS

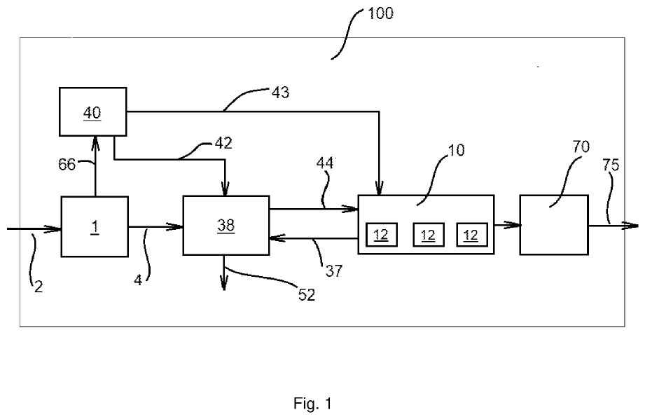

Referring to FIGS. 1, 2 and 19, there is shown a block diagram of a system 100 for generating and using carbon dioxide to produce a biofuel, in particular Biodiesel (a fatty acid methyl [or ethyl] ester), and/or Renewable Diesel (a paraffin) and/or other paraffinic fuels such as Sustainable Aviation Fuel. The system 100 comprises a “closed system combustion” stage 38 containing one or more “closed system furnace/s” that generates carbon dioxide 44 for consumption in the “closed algae growth and oxygen generation” stage 10 and produces cogeneration heat and/or steam to:
a. power a steam turbine electrical generator 190; and/or
b. distill ethanol beer 42 to an ethanol rich solution 52; and/or
c. to evaporate 63 juice to molasses 64; and/or
d. to preheat ethanol 52 for ethanol vapour permeation process 54; and/or
e. to preheat lipids for hydrogen processing; and/or
f. to preheat ethanol 55 for dehydration into ethylene; and/or
g. to preheat product ethylene gas for downstream processing into polyethylene and/or sustainable aviation fuel; and
   a “closed system fermentation” stage 40 for fermenting a sugar containing material to generate ethanol beer 42 and carbon dioxide 43; and a “closed algae growth and oxygen generation” stage 10 requiring carbon dioxide as an input to a process requiring carbon dioxide.

In this embodiment, a “proximate crop processing plant” stage 1 is provided prior to the closed system combustion stage 38 and closed system fermentation stage 40 for proximate processing of a crop of sugar imbued plants, here sweet sorghum. Although this embodiment is most advantageous, it does not preclude other sugar imbued feedstocks, or remote processing, (for example it may include sugar cane processed at a centralised mill) for use in “closed system fermentation” stage 40.

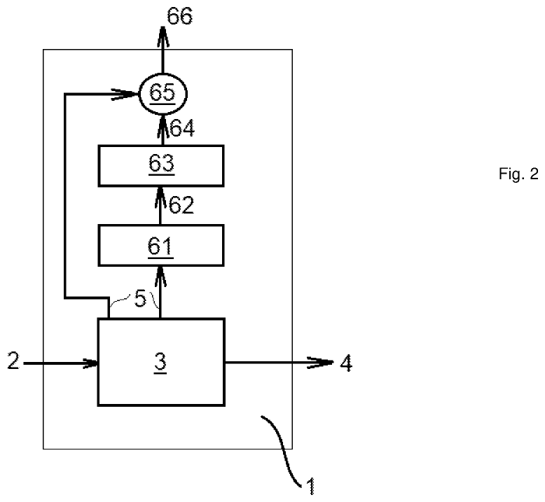

The “proximate crop processing plant” stage 1 preferably includes a crusher 3 (with reference to FIG. 2) to reduce the sugar imbued crop 2, to juice 5 and bagasse 4. The sugar imbued crop will contain a high content of water and sugar typically in excess of 60% by weight, but once crushed and the juice removed, the bagasse 4 will typically be about 40% to 55% by weight moisture. This level of moisture content does not stop the bagasse 4 from sustaining combustion, and it can be passed directly (or indirectly if the bagasse 4 is stockpiled) to the “closed system furnace” 38 (the “closed system combustion” stage) which preferably is a chain grate and/or fluidised bed and/or blast sealed combustion furnace boiler.

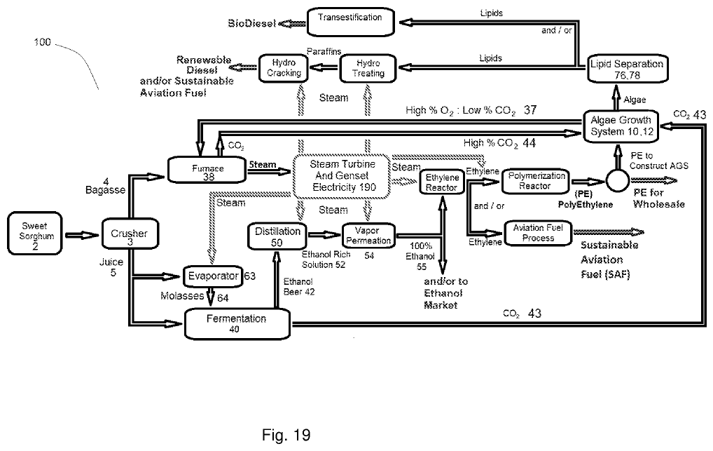

The furnace of closed system combustion stage 38 (FIGS. 1 and 19) is dimensioned to handle the bagasse 4 sourced from stockpiles or fed directly to a crusher, and is a closed system preferably intolerant to gas leakage and from which gas streams can be controllably directed to other stages of the system 100. The furnace burns the bagasse 4 in a gas 37 containing high concentrations of oxygen and the remainder carbon dioxide sourced from the closed algae growth and oxygen generation stage 10. This in-turn enables the production of high concentrations of CO₂ 44 to be supplied to the closed algae growth and oxygen generation stage 10 and in the case of CO₂ 44 from the closed system combustion stage 38 after being passed through a heat exchanger to be cooled before supplying to the closed algae growth and oxygen generation stage 10. In this regard, combustion gas is hot and its temperature must be reduced to avoid destruction of algae in closed algae growth and oxygen generation” stage 10 and more desirably at a temperature within that optimal for algal growth.

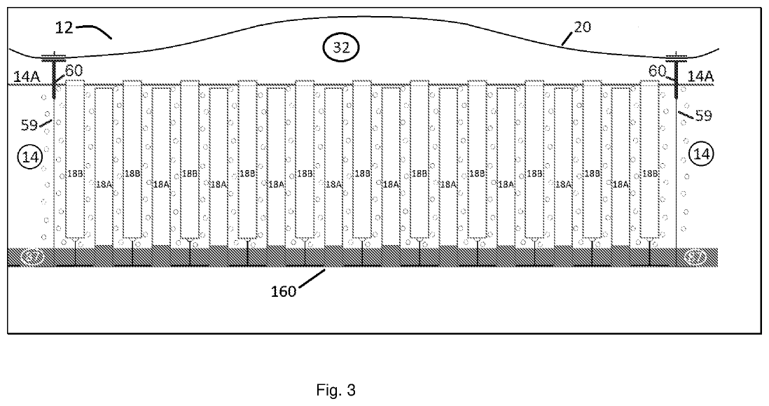

Air is preferably not used as a combustion gas. The closed algae growth and oxygen generation 10 sub-system's multi-panelled sealed tent(s) 12 operate more efficiently using high concentrations of CO₂ feedstock, than gases resulting from the combustion of bagasse 4 in air (containing principally oxygen and nitrogen). With respect to air, CO₂ storage within the closed algae growth and oxygen generation system 10 will be adversely impacted if gases containing carbon dioxide and oxygen, but also containing approximately 78% by volume nitrogen are utilised—as would be the case with air—and which will poach space with little benefit to the purpose of the CO₂ storage facility 32 in FIG. 3.

Carbon dioxide is effectively inert in the closed system combustion stage 38 combustion gas and passes through that system to enrich the carbon dioxide as produced by the closed system combustion stage 38 to be delivered back to the closed algae growth and oxygen generation stage 10. If air was used as a feedstock to the closed system combustion stage 38, the nitrogen (78% by volume) would oxidise to produce some NOₓ gases but would in the main be also inert, both to the closed system combustion stage 38 and the closed algae growth and oxygen generation stage 10. However, by configuring the closed system furnace 38 to burn at sufficiently high temperatures and with high oxygen concentrations, a measured amount of nitrogen can be introduced to produce NOₓ gas to be used as a fertiliser for the algae medium. Air (i.e. 78% Nitrogen) would, in the main, occupy valuable storage space and is therefore not promoted as a component of the combustion gas of the closed system combustion stage 38.

Closed algae growth and oxygen generation stage 10 here involves cultivation of algae for the purpose of producing biofuel. The algae may, in principle, be any type of waterborne microalgae that requires light energy (e.g. sunlight) and carbon dioxide for growth with formation of lipids convertible to biofuel. However, high lipid content is preferred and—in some embodiments—the algal or microalgal strain may require NOx and SOx tolerance. Chlorella spp may be one suitable selection, for example Chlorella vulgaris, an algal species with characteristics much studied in the art.

While algae are used for the present description, it will be appreciated that—in other embodiments—closed algae growth and oxygen generation stages 10 using alternative, or additional, carbon dioxide respiring organisms or life forms and for producing chemical products other than the production of biofuels are included within the scope of embodiments of the invention.

### Crop Selection and Proximate Crop Processing Plant

Particular varieties of Sorghum bicolor L Moench, known as ‘sweet sorghums’, accumulate large amounts of sugar in their stems. Near the time of grain maturity, sweet sorghums typically have 10 to 25% by weight sugar in the stalk juice, with glucose/fructose being the predominant disaccharide. Sweet sorghum R9188 can provide an average Brix of 13%.

Sweet sorghum is a fast growing grass, with low water consumption and suitable for growth in seasonally arid regions, that has high carbon dioxide uptake. A single crop of sweet sorghum is capable of providing about 80 Tonnes of sweet sorghum per hectare, and because it is fast growing, and depending on water availability, is able to provide two or even three crops per year which facilitates balancing of CO₂ generated during the closed system combustion stage 38 and/or closed system fermentation stage 40 with requirements for algal growth in closed algae growth and oxygen generation stage 10 as described above. Further, in the case of two crops of sweet sorghum per year, it is calculated that about 82 Tonnes of CO₂ are consumed per hectare from the atmosphere, representing a very efficient CO₂ sequestration system, about four times greater per hectare than fast growing tree species such as Blue Gum Eucalyptus (as grown on timber plantations in about ¼ million hectares of the South West of Western Australia).

Historically, a sugar crop has been crushed using “large scale centralised mill facilities”. In embodiments of the present invention, crushing of harvested sweet sorghum 2 by “proximate crop processing plant” 1 crusher(s) 3 allows farm scale crushing of sweet sorghum (or sugar cane) 2 to produce juice 5 used in different forms as feedstock for the closed system fermentation stage 40 in proximity to the field without requirement of a “large scale centralised mill facility”. In the context of this application, “a large scale centralised mill facility” is typified using Queensland, Australia's 1994 production from its 25 centralised mills operating at the time and which crushed 32,846,617 tonnes of sugar cane averaging 1,314,000 tonnes of cane per “large scale centralised mill facility”.

In contrast, a “proximate crop processing stage's” crusher 3 is dimensioned for processing approximately 80,000 tonnes of sorghum per year and is approximately 16 times smaller than the sum of crushers used in a “large scale centralised mill facility” as described above.

Electrical power for the crusher 3 is supported by a steam turbine generator 190 and/or one or more integrated proximate genset/s, all collectively capable of producing greater than 500 kW of power. Local production of bagasse 4 in proximate crop processing plant 1 reduces bagasse transportation costs, to a closed system furnace that can suitably reticulate carbon dioxide to, and be balanced in scale with, the requirements—driven by algal carbon dioxide uptake—of closed algae growth and oxygen generation stage 10.

The constituency of sweet sorghum is typically as follows (% by weight):

| | |
|---|---|
| Sugars | 17% |
| Bagasse | 16% |
| Water | 64% |
| Balance | 3% |

For example, the quantity of bagasse material 4 obtained from the crushing of a harvest of about 80 tonnes per hectare crop of sweet sorghum is about 12.8 tonnes per hectare of bagasse 4. As well as the bagasse 4 being burned in the furnace(s) of the closed system combustion stage 38 in the production of CO₂ 44, its primary function, the heat and steam generated within the boiler of the furnace of closed system combustion system 38 allows recovery of sufficient cogenerated heat to be utilised in many of the system 100 processes, including but not limited to, distilling a primary ethanol beer 42 from the closed system fermentation stage 40 in the closed system combustion stage 38 boiler to produce an ethanol rich solution 52.

As shown in FIG. 2, distillation using heat exchangers 63 energised by waste heat recovery from the furnace(s) of the closed system combustion stage 38 and/or optional ultra-filtration together with reverse osmosis system/s 61 that are optionally contained in the “proximate crop processing plant” 1 minimise the volume (and storage) of juice 5 by thickening it into molasses 64 and/or 62 (which enables a higher ethanol content to be produced in the closed system fermentation stage 40, reduces juice storage facility requirements, reduces capital costs and prolongs the life of the juice by thickening for use in later fermentation). Molasses 64, together with a low sugar content solution such as the portion of juice 5 not processed to molasses, is mixed at 65 and directed as a sugar containing stream 66 to the fermentation still(s) of closed system fermentation stage 40.

### Furnace of Closed System Combustion Stage 38

Chain grate and/or fluidised bed and/or blast sealed combustion furnace(s) is/are preferred for closed system combustion stage 38. The furnace(s) burn(s) bagasse 4 obtained from a stockpile or crusher 3 as described above.

Ash from burnt bagasse 4, which may be referred to as “mill mud”, may conveniently be used as a fertiliser for sweet sorghum farming.

The furnace of combustion stage 38 will burn the solid plant material 4 in a gas containing high grade oxygen and the remainder carbon dioxide 37 (as provided by the closed algae growth and oxygen generation system 10) to produce high grade CO₂ 44 that is captured within the furnace and passed through a heat exchanger for regulated delivery of CO₂ feedstock 44 to the algae multi-panelled sealed tents 12 of the algae growth and oxygen generation system 10.

A heat exchanger/s is a component of the closed system combustion stage 38 and is used to remove heat from the CO₂ exhaust of that stage 38, suitable for delivery of the gas 44 at desired temperature, as described above, to the closed algae growth and oxygen generation stage 10.

Furthermore, heat reticulated as combustion gas or steam from the closed system combustion stage 38 can be recovered and used via additional heat exchanger(s) to:
a. power a steam turbine electrical generator 190; and/or
b. distill 50 ethanol beer 42 to an ethanol rich solution 52; and/or
c. to evaporate 63 juice to molasses 64; and/or
d. to preheat ethanol 52 for ethanol vapour permeation process 54; and/or
e. to preheat lipids for hydrogen processing; and/or
f. to preheat ethanol 55 for dehydration into ethylene; and/or
g. to preheat product ethylene for downstream processing into polyethylene and/or sustainable aviation fuel.

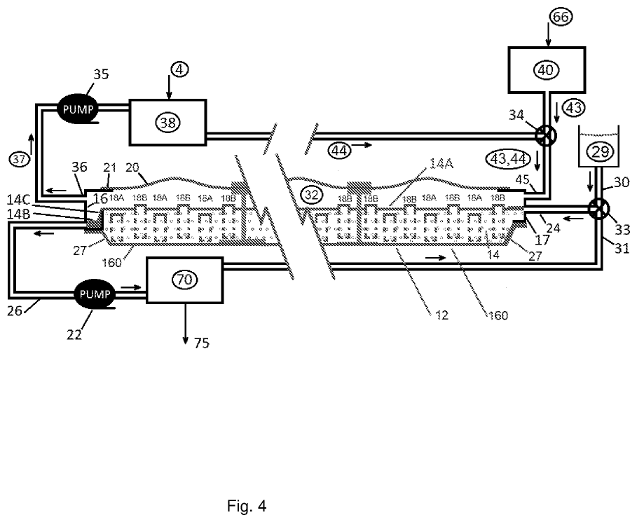

During growth of algae, oxygen is produced through algal photosynthesis. This oxygen is trapped under the seal or ceiling 20 of the multi-panelled sealed tent 12 (refer to FIGS. 3 and 4) and may be collected at a gas offtake 36 to function as the oxygen supply 37 and delivered by pump 35 to the closed system combustion stage 38. This oxygen 37 will contain CO₂, by virtue of the storage facility 32 also containing CO₂.

The gas 37, being in the main for reasons described above, a nitrogen limited mixture of high grade oxygen and the remainder carbon dioxide, is buffered within the multi-panelled sealed tent 12 in the CO₂ storage facility 32 (FIGS. 3 and 4) in that the CO₂ storage facility space 32 can be dimensioned to store several days supply of CO₂ in the presence of approximately 60% oxygen by volume. This allows the closed system combustion stage 38 to operate at a burn rate that is compatible with the average CO₂ 44 uptake rate of the waterborne algae in the algae medium 14.

Because of the recirculation of gases between the closed system combustion stage 38 and the closed algae growth and oxygen generation stage 10, an imbalance of gas production/consumption may—as observed above—ultimately raise the concentration of O₂ in furnace(s) of the closed system combustion stage 38 to concentrations of O₂ in excess of 60%, which would increase the rate of fuel burn in the furnace(s) of the closed system combustion stage 38 thereby increasing the temperature of the fuel burn. Such temperature may be problematic for retrofitting into such furnace systems, as found for example in power stations. An option for addressing such issues is to reduce the dimensions of the closed algae growth and oxygen generation system 10 such that it cannot consume all of the CO₂ as limited by algae growth conditions and delivered from the closed system combustion stage 38. Thereby, the algae physiology and growth conditions themselves pro-rate the CO₂ consumption down and likewise the O₂ production down. Then, given the sustained production of CO₂ from the closed system combustion stage 38, the surplus CO₂ not used by the closed algae growth and oxygen generation stage 10 increases the CO₂ concentration in the recirculating gas. Thus, by increasing or reducing the number of multi-panelled sealed tent(s) 12 operational in the closed algae growth and oxygen generation stage 10, the O₂ concentration in gas directed to the furnace(s) of the closed system combustion stage 38 and the fuel burn temperatures can be controlled as a closed system. Excess CO₂, in excess of algal growth requirements, can be controlled by increasing the number of multi-panelled sealed tent(s) 12 operational in the algae growth and oxygen generation stage 10. The excess O₂ may be vented or commercialised where there is an available market.

### Optional Use of a Steam Turbine for Electrical Generation 190

The primary function of the closed system combustion stage 38 is to produce CO₂ 44 for the closed system algae growth and oxygen generation stage 10 which receives the carbon dioxide 44 generated in the closed system combustion stage 38. The byproduct of this CO₂ production is the large amount of heat energy generated, which is best dissipated as steam to drive a steam turbine electricity generator 190 with remnant steam then being reticulated through heat exchangers that condense the steam back to hot water (thus producing a vacuum pulling the steam through the turbine 190, with the effect of a “condensing turbine”). The heat exchangers are used but not limited to applications such as the following processes of distillation, molasses evaporation, ethanol vapour permeation, dehydration of ethanol to ethylene, polymerization of ethylene and hydroprocessing of lipids. Electrical energy produced by the steam turbine electricity generator is used by the majority of system components.

### Blending of Sweet Sorghum Juice and Molasses

In this embodiment, target sugar content of the sugar containing material—here a blend 66 of juice and molasses produced as described above—is about 23.5 Bx. As sweet sorghum juice would typically have insufficient sugar concentration to provide a target ethanol concentration of 12 vol %, it is blended 65 with the molasses thereby allowing the fermentation process in closed system fermentation stage 40 to reach the target ethanol concentration. The higher the concentration of ethanol, the less the energy input required for the distillation process 50 per tonne of ethanol. It is to be understood that the 12 vol % target is by way of example and if, for example, it becomes possible to achieve higher ethanol contents through advances in fermentation technology, that higher potential target ethanol concentration is intended to fall within the scope of embodiments of the invention.

### Fermentation Stage 40

Closed system fermentation stage 40 comprises one or more stills for fermenting a sugar containing juice, a juice/molasses blend and/or a juice concentrate with a suitable yeast, such as S cerevisiae spp, to produce the primary ethanol beer 42 and CO₂ 43 which is directed to one or multiple algae multi-panelled sealed tents 12 in the closed algae growth and oxygen generation stage 10 where it is balanced (in conjunction with CO₂ 44 from the closed system combustion stage 38) with the growth requirements of waterborne algae (such as C. vulgaris) as described above. Suitable fermentation stills are well known in the art of ethanol production and are not further described here.

Juice and molasses 66 produced from a sweet sorghum, are directed to closed system fermentation stage 40 which has an ethanol target of 12 vol % as described above in paragraph [00133]. For the purpose of example, assuming a sugar content of about 50 wt % from molasses production, and a juice sugar content of 17 wt %, a ratio of about 80 wt % juice and 20 wt % molasses input to the closed system fermentation stage 40 will achieve the necessary 23.5 wt % sugar content requirement to achieve, with full fermentation, a 12 vol % ethanol beer 42. It will be understood that these sugar contents, ratios of juice/molasses or juice: concentrated juice ratios and ethanol contents are provided by way of example and other sugar contents, juice: molasses ratios and ethanol contents are feasible.

### Distillation of Ethanol Using the Boiler in the “Closed System Furnace”

A 12 vol % ethanol beer 42 is too dilute to be commercially viable as a product and therefore needs to be distilled 50 towards the limit of azeotropic water content (typically 4.4% water) thus theoretically producing approximately 95% ethanol rich solution 52, which is commercially viable.

In practice, the ethanol rich solution 52 will be lower than the theoretical azeotropic limit and thus a membrane vapor permeation system 54 can be used to increase the ethanol rich solution 52 concentration to above 99% ethanol purity 55.

The net calorific value (NCV) of bagasse 4 resulting from the above crushing (drying) process, and assuming a moisture content of 50% moisture, is about 8 MJ/kg. With 54% moisture content this is reduced to about 7 MJ/kg. Net Calorific Value (NCV) is the energy available after compensating for the energy absorbed to evaporate the water off the bagasse 4.

By way of example, assuming an NCV of 7 MJ/kg, there is about 89,000 MJ of net calorific energy available in 16% wt content bagasse 4 per hectare per harvest of sweet sorghum 2 (assuming 80 Tonnes of sweet sorghum/hectare/harvest), enough to distill about 30 Tonnes/hour year-round of 12% ethanol beer 42 (assuming 100% efficiency) from an approximate 500 hectare sweet sorghum farm when the distillation requirement 50 is only about 2.5 Tonnes of 12% ethanol beer 42 per hour, year round from the same farm. Thus the furnace(s) of the closed system combustion stage 38 has sufficient energy output to produce steam and reticulate to heat exchangers for the distillation 50 of an ethanol rich solution 52.

With a crop balanced with the throughput of a “proximate crop processing plant” 1, the CO₂ generation within the closed system combustion stage 38 and closed system fermentation stage 40 and the oxygen generation 37 from the closed algae growth and oxygen generation stage 10, the system 100 can produce a significant amount of biofuel. A portion of biofuel can be used for power requirements of farming and processing the selected crop, for instance sweet sorghum. Power may be generated by biofuel and biofuel powered gensets, where the biofuel can be Renewable Diesel or other paraffinic fuels, Biodiesel and/or Ethanol. This biofuel can be used to supplement electrical and farm equipment power needs and used to power the system 100 equipment including but not limited to closed algae growth and oxygen generation stage 10, algae harvesting and downstream processing 70, closed system fermentation stage 40, “proximate crop processing plant” 1, closed system combustion stage 38, membrane filtration 61, evaporation systems 63 and distillation systems 50, and control systems (here a SCADA system for controlling system 100, though other forms of control system can be used) and other process equipment.

### Closed Algae Growth and Oxygen Generation Stage 10

Closed algae growth and oxygen generation stage 10 includes a means for storing carbon dioxide as part of balancing carbon dioxide generated by combustion of fuel in the furnace(s) of the closed system combustion stage 38 with that respired by the algae in the closed system algae growth and oxygen generation stage 10. In this embodiment, a gas storage facility 32 (FIG. 3), in the form of the enclosed space between the liquid algae growth medium water level 14A, and the top impervious translucent ceiling sheet 20 of each multi-panelled sealed tent 12 panel, serves to balance the generation of carbon dioxide from both the closed system combustion stage 38 and the closed system fermentation stage 40 with carbon dioxide requirements of the waterborne algae in the closed algae growth and oxygen generation stage 10. The gas storage facility 32 and closed algae growth and oxygen generation stage 10 are described in detail below.

Closed algae growth and oxygen generation stage 10 involves one or typically a plurality of vessels, in this embodiment in the form of closed and multi-panelled sealed tents 12, for growing algae. A large number of multi-panelled sealed tents” 12, potentially many hundreds of multi-panelled sealed tents 12 could be included dependent on biofuel production targets. A cross section of a panel contained in a multi-panelled sealed tent 12 is shown in FIG. 3 and a long section of a multi-panelled sealed tent 12 is shown in FIG. 4 and described, for purposes of exemplification, below. Growth of algae in closed algae growth and oxygen generation stage 10 requires light energy, carbon dioxide, nutrients and a growth medium.

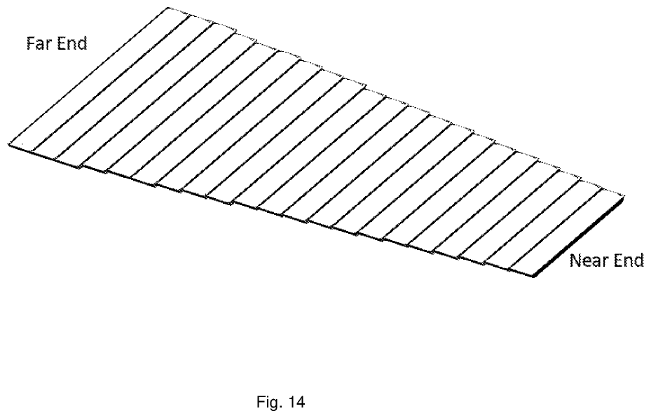

A rendered multi-panelled sealed tent 12 is also shown by way of example in FIG. 14, with the translucent ceiling sheet 20 constructed using rolls of manufactured sheets (for example 7 m sheets) cut to length and that are clasped as described below on all four sides to establish a sealed ceiling 20.

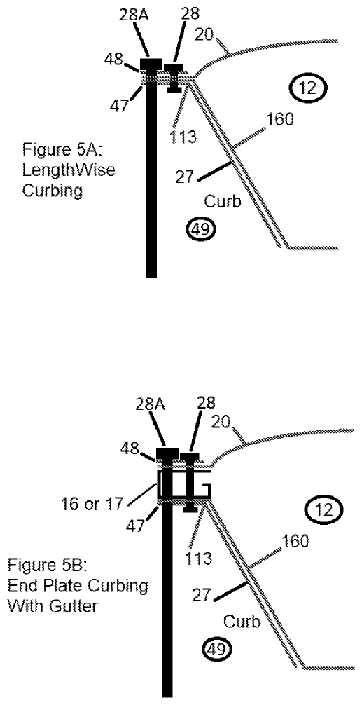

Each multi-panelled sealed tent 12 comprises a floor 160 and translucent cover (seal or ceiling) 20, supported on the four sides of the multi-panelled sealed tent 12 using curbing 27 (FIG. 5A,5B). Each multi-panelled sealed tent 12 is terminated at each end with respective end plates 16, 17. The “far end” plate 16 effects the removal of liquid algae growth medium 14 and an oxygen/carbon dioxide gas mixture 37 from the “multi-panelled sealed tent” 12 and the “near end” plate 17 supplies liquid algae growth medium 14 and carbon dioxide gas 43,44 into the multi-panelled sealed tent 12.

A multi-panelled sealed tent 12 for the purpose of growing algae, contains an algae growth medium 14 comprising a liquid suitable for supporting algal growth. In particular, the growth medium 14 comprises water which is contained in and constrained by the multi-panelled sealed tent 12, having a surface water level 14A within which waterborne algae flow (i.e. move) from a “near end” plate 17 of the multi-panelled sealed tent 12 to the “far” end plate 16. The multi-panelled sealed tents 12 may be located within a construction or excavation, for example, to provide a supporting structure and reduce the height of the multi-panelled sealed tent 12 above ground.

The multi-panelled sealed tent 12 is a closed system in which algae are grown in isolation from airborne pollutants and stray algal cells and which allows controlled gas flows within it and to and from the closed system combustion stage 38.

Closed algae growth and oxygen generation stage 10 further comprises a transparent seal or ceiling 20 for closing and sealing the multi-panelled sealed tent 12; a pump 22 for moving the liquid algae growth medium 14 bearing algae throughout the multi-panelled sealed tent 12; and an inlet 24 for recirculating liquid algae growth medium 14, replacing water consumed by algae and/or lost to the process, injecting or otherwise delivering or introducing matter, such as nutrients, promoting algal growth through the “near end” plate 17 of the multi-panelled sealed tent 12.

The material of the transparent or translucent seal 20—and any other portions through which sunlight is to travel—desirably include UV stabilisers and other chemical additives to constrain the wavelength of light transmitted into the multi-panelled sealed tent 12. For example, the green portion of the light spectrum does not deliver light conducive to algal growth so an additive such as a dye (desirably pink in colour) may be used to exclude the green portion of the visible light spectrum.

The seal 20 comprises a sheet of flexible translucent material sealed on each side of the multi-panelled sealed tent 12 and at each end using compressive forces where:
a. on each long side of the multi-panelled sealed tent 12 between a base plate 47 on which the seal 20 and base 160 sheets are preferably folded and clamped together between a clamping strip 48 and the base plate 47 as shown in FIG. 5A. A clamping bolt 28 is used clamp the base plate 47 and clamping strip 48 together; and
b. on each end plate 16,17 and using a compression fixing device such as a compression band or clamping strip 48 to fix seal sheet 20 to the “near end” and “far end” plates 16,17 of the multi-panelled sealed tent 12. Likewise, the floor (and sides) 160 of the multi-panelled sealed tent 12 are similarly clamped to each end plate 16,17 using preferably a base plate 47 or compression devices as shown in FIG. 5B; and
c. the compressive forces make the multi-panelled sealed tent 12 airtight while allowing the energy source for the algae (sunlight, in this embodiment) to radiate into the liquid algae growth medium 14 and also be captured by the Light Diffusion Device(s) 18A, 18B (herein “LD Devices” and generically known as “18”) for light distribution at depth in the liquid algae growth medium 14 of the multi-panelled sealed tent 12.

The curbing 27 of FIG. 5A,5B fulfills a number of functions, in that
a. it supports the multi-panelled sealed tent 12 laterally;
b. it holds the multi-panelled sealed tent 12 at design height;
c. it secures the base plate 47 to the multi-panelled sealed tent 12; preferably by bending a sheet, for example of polyethylene, forming the curbing 27 and at the top of the curbing, which is flat, to then form the base plate 47;
d. it then enables two sheets (floor sheet 160 and ceiling sheet 20) to be clasped together for the length of the multi-panelled sealed tent 12 to create a seal as depicted in FIG. 5A; and
e. at the near and far ends, it can support the end plates 16,17, which can be sandwiched between the base plate 47 and the clamping strip 48 with the clamping strip 48 securing the ceiling sheet 20 against the roof of the end plate 16,17 and the base plate 47 securing the floor sheet 160 against the floor of the end plate 16,17 to provide an effective airtight seal as depicted in FIG. 5B;
f. it is itself supported by earthworks or similar structure 49, providing a clean interface between the supporting structure 49 and the floor (and sides) 160 of the multi-panelled sealed tent 12;
g. it can secure the ceiling sheet 20, floor sheet 160, baseplate 47 and clamping strip 48 to the supporting structure 49 by way of a grounding stake 28A to provide fixed foundational support against movement associated with wind and other external influences.

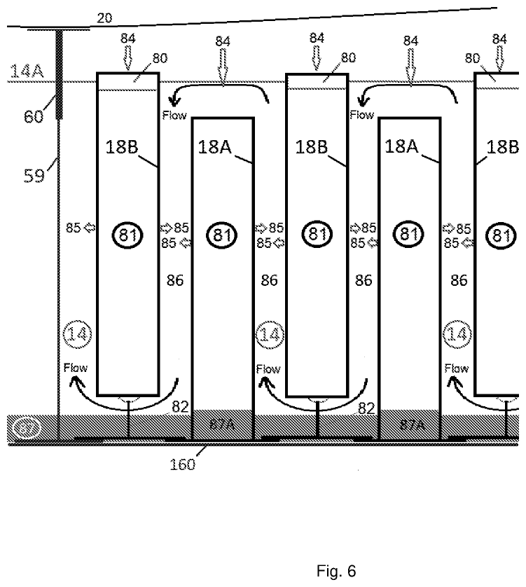

Contained in the multi-panelled sealed tent system 12 are one or more Light Diffusion Device(s) 18A, 18B (herein “LD Device(s)” and generically numbered as “18”) in the form of translucent sealed containers as shown in FIG. 6.

LD Device(s) 18A, 18B capture light 84 above or about the surface 14A of the liquid algae growth medium 14 and diffuse that light through water 81 contained in the LD Device(s) 18A, 18B separate from the algae growth medium. The water 81 is preferably doped with light reflective material to enhance light diffusion.

The light reflective material in the clarified water 81 may, for example, be a combination of a Florescent Brightener and a dye. A pink dye is preferable as it provides some blue spectrum with the red spectrum at the exclusion of green. Green spectrum is emitted by algae and not useful to photosynthesis.

The water 81 in each LD Device 18A,18B is preferably initially sterilised and clarified for example using membrane technology and is conveniently permanently stored in the LD Device 18A,18B. The water 81 provides sufficient hydrostatic pressure to counter the hydrostatic pressure of the liquid algae growth medium 14, and assist the LD Device(s) 18A, 18B to keep their shape.

The LD Device(s) 18A, 18B are sealed at each lateral end with a water and airproof seal, to permanently store water in the LD Device 18A, 18B and in the case of the “In-filler” LD Device 18B to capture air for buoyancy. Air, in the case of LD Device 18B and water in the LD Devices 18A, 18B can be injected/removed using a hose(s) that is sealed and fixed into the end of the LD Device 18A, 18B.

The shape of each “In-filler” LD Device 18B is maintained by injecting some air 80 in each LD Device 18B, that occupies the space between the surface of the water 81 and the top of the LD Device 18B providing it with buoyancy.

The use of air in LD Device 18B is preferred to CO₂ (which is used in the CO₂ storage facility 32), as air is not as soluble as CO₂ and avoids the buildup of carbonic acid in the water 81.

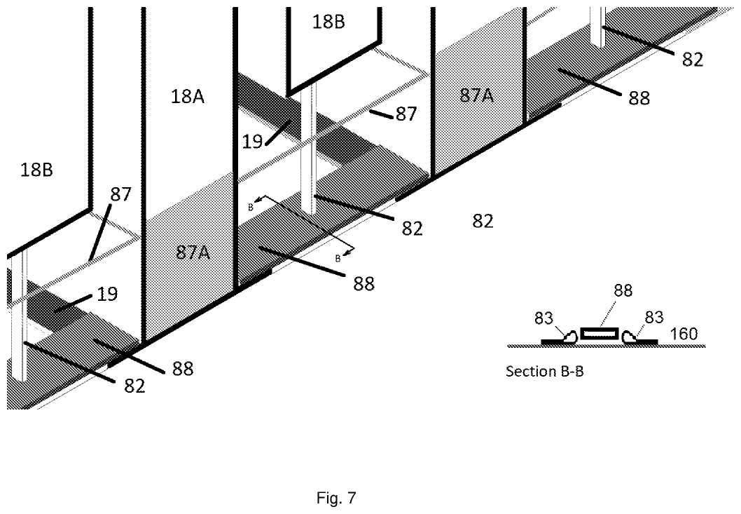

Spacing between LD Device 18A Braced Rows is maintained with a spacing bar 88 (FIG. 7) which can be secured to the extended base plates 19 of adjacent LD Device 18A Braced Rows. The spacing bar 88 can also be strapped to the floor 160 of the “multi-panelled sealed tent” 12, preferably using tethers 83 which are welded to the floor 160.

The LD Device 18A Braced Row, is also partially submerged beneath optional Sand Ballast 87 that covers the top of the extended base plate 19 of the LD Device 18A, and by virtue of the Sand Ballast 87 weight assists in securing each LD Device 18A Braced Row in position. The sand ballast may be sterilised.

The LD Device 18A Braced Row also preferably contains ballast 87A at the base of the container which again provides stability and further security of position.

Buoyancy and movement of the LD Device 18B “In-filler” is countered using a tether 82 which secures the LD Device 18B “In-filler” container to the spacing bar 88 which can be further secured to the extended base plates 19 of adjacent LD Device 18A Braced Rows and by using tethers 83, the spacing bar 88 can be further secured to the floor 160 of the “multi-panelled sealed tent” 12. It will be understood that securing means other than tethers 82 could be used.

Exemplary to the invention, and without limitation, if the LD Device(s) 18A, 18B are constructed with approximately 3 mm UV protected translucent plastic material (such as for example: PE (PolyEthylene), PET (Polyethylene Terephthalate), PMMA (PolyMethyl MethAcrylate) or other suitable plastics that are commercially available), then the LD Device(s) 18A, 18B will have sufficient rigidity, and allow the containers to stand supported by the algae medium.

Each LD Device 18B “In-filler” container is buoyant (see FIG. 6) resulting from air 80 captured at the top of the LD Device 18B “In-filler” container and can be purposefully floated a distance from the floor 160 (or optional sand ballast 87 covering the floor 160) and preferably at a distance of 5 cm to 10 cm, to achieve two aims:
a. to allow the movement of water underneath the LD Devices 18B “In-filler” container and thereby between algae growth passages 86, where an algae growth passage 86 (FIG. 6) is that space between LD Device(s) 18A, 18B; and
b. for the tethers 82 to provide spacing between the LD Device 18B “In-filler” container and floor 160 (or optional sand ballast 87 covering the floor 160) so that the LD Device 18B will clear the rope/chord 94 and base of LD Device Cleansing System 96 as described below, thereby reducing the wear or interfere with the LD device 18B.

The dimensions of the LD Devices 18A, 18B are selected as a function of available light energy. Sunlight can be measured in moles of photons and without wishing to be bound by theory, typical noonday sunlight in Northern Australia is approximately 1,700 μmol·photons/m²/sec. However, the optimum irradiance for a wide range of algae species is typically between 120 to 400 μmol·photons/m²/sec which is a small fraction of the sun's radiation.

Therefore an average quantity of light 85 emitted from the sides of the LD Devices 18A, 18B can be calculated, assuming an absorption rating for the combination of translucent ceiling sheet 20 and LD Devices 18A, 18B, applied to the expected sunlight radiation energy, and when the red and/or blue Photosynthetically Active Radiation (“PAR”) spectrums are separated (for use by the algae), an average irradiation energy 85 from the sides of the LD Device 18A, 18B can be computed to assist in determining the dimensions, both width and depth, of the LD Device 18A, 18B.

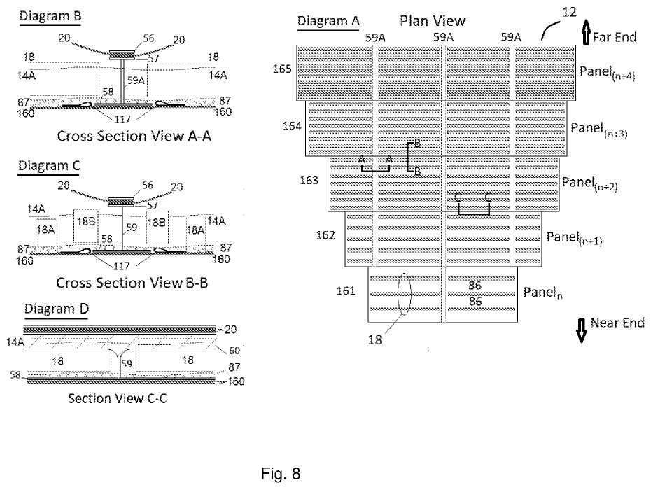

The extent of the light pathway from the sides of the LD Devices 18A, 18B through the liquid algae growth medium 14 can be approximated using a linear interpolation of algae density using as a basis the penetration of light limited to, for example, 5 cm at 1.5 gm/Litre (as described in Raeisossadati (2020) Luminescent solar concentrators to increase microalgal biomass productivity; PHD Thesis; Murdoch University, WA, Page 62 and 80 and the contents of which are hereby incorporated herein by reference). This enables a calculation of the separation between LD Devices 18 in the multi-panelled sealed tent(s) 12 and, by way of example, is as per the following table, for 20 cm wide LD Devices in approximately 1.7 m water and arranged in 6.5 m wide panels within the “multi-panelled sealed tent” as schematically described in FIG. 8, Diagram A:

| | | | | | Panel | |
|---|---|---|---|---|---|---|
| | | | | Number of | location | Panel |
| Algae | | Light | | LD Devices | lengthwise | Length |
| Density | | Penetration | | across panel | (m) in Tent | m |
| 1.57 | gm/L | 4.5 | cm | 21 | 130.0 | 100 |
| 1.50 | gm/L | 5.0 | cm | 21 | 123.5 | 100 |
| 1.42 | gm/L | 5.0 | cm | 21 | 117.0 | 100 |
| 1.35 | gm/L | 5.5 | cm | 20 | 110.5 | 95 |
| 1.28 | gm/L | 6.0 | cm | 20 | 104.0 | 90 |
| 1.20 | gm/L | 6.0 | cm | 20 | 97.5 | 90 |
| 1.13 | gm/L | 6.5 | cm | 19 | 91.0 | 86 |
| 1.06 | gm/L | 7.0 | cm | 19 | 84.5 | 83 |
| 0.98 | gm/L | 7.5 | cm | 18 | 78.0 | 80 |
| 0.91 | gm/L | 8.5 | cm | 17 | 71.5 | 75 |
| 0.84 | gm/L | 9.0 | cm | 17 | 65.0 | 73 |
| 0.76 | gm/L | 10.0 | cm | 16 | 58.5 | 70 |
| 0.69 | gm/L | 11.0 | cm | 15 | 52.0 | 67 |
| 0.62 | gm/L | 12.0 | cm | 14 | 45.5 | 64 |
| 0.54 | gm/L | 13.5 | cm | 13 | 39.0 | 61 |
| 0.47 | gm/L | 16.0 | cm | 12 | 32.5 | 57 |
| 0.39 | gm/L | 19.0 | cm | 11 | 26.0 | 54 |
| 0.32 | gm/L | 23.0 | cm | 9 | 19.5 | 51 |
| 0.25 | gm/L | 30.0 | cm | 8 | 13.0 | 48 |
| 0.17 | gm/L | 43.0 | cm | 6 | 6.5 | 44 |
| 0.10 | gm/L | 75.0 | cm | 3 | 0.0 | 44 |

Each panel, 161, 162, 163, 164, 165 (being a subset of all panels) has a uniquely defined growth passage 86 width compatible with the algae density interpolated for that panel. Each panel 161-165 also has approximately the same volume of liquid algae growth medium as its adjacent panels and hence there is varied panel length resulting from the different number of LD Devices 18 assigned to each panel 161-165.

In the above example, and not to limit this invention, three LD Devices 18A, 18B would be required, across the panel proximate to the “near end” plate 17 of the “multi-panelled sealed tent” 12 where the algae concentration is the seed concentration (i.e only about 0.10 gm/L). At the other end of the multi-panelled sealed tent 12, in the 21^{st} panel, there are twenty one LD Devices 18 proximate to the “far end” plate 16 where the algae concentration is at the design harvest concentration of about 1.57 gm/L.

Because the length of the panels 161-165 may be considered excessive, then it is possible to section the panels using section brace(s) 59A (as shown in FIG. 8 Diagrams A and B). For example, if a 20 m panel section length was desired, then the same approximate length of ceiling sheet 20 can be joined to a subsequent length of ceiling sheet 20 in continuation of the panel by bracing the two ceiling sheets 20 between a clamping strip 56 and a base plate 57 that is fixed to the panel section brace 59A which in turn, is mounted on a floor plate 58.

This then allows the LD Devices 18 to be manufactured in manageable lengths suitable for a panel section.

The above example is schematically shown in FIG. 8 Diagram A where five panels 161, 162, 163, 164, and 165 of the multi-panelled sealed tent 12 are schematically shown, demonstrating different layouts for each panel, of LD Devices 18 (18A, 18B) where the uniquely defined growth passage 86 width is commensurate with the algae density as interpolated from “Near End” to “Far End” for each panel 161-165.

If, in the above example, the movement of algae is controlled with an inflow of liquid medium 14 into the multi-panelled sealed tent 12 through the inlet 24 of approximately 15 m³/hr and a similar outflow at the outlet 26, it will take 16 days to traverse the multi-panelled sealed tent 12 (the life span of algae is about that duration). This implies that the maximum flow rate over a LD Device 18A Braced Row is about 0.6 cm/sec assuming a 2 cm deep aperture of water flowing over the LD Device 18A.

The flow of liquid algae growth medium, described in FIG. 6, is enabled by LD Device(s) 18B “In-filler” containers desirably purposefully floated at a sufficient distance from the floor, to allow the movement of water underneath the LD Device 18B, whereas the LD Device(s) 18A Braced Rows are secured by bracing to the floor of the multi-panelled sealed tent 12 and the tops of which are below the algae medium water level 14A, allowing the medium to flow over the LD Device(s) 18A Braced Rows. Thereby, as an alternate pattern of containers 18A and 18B, the flow of liquid algae growth medium is sequentially directed over the LD Device(s) 18A “Braced Rows”, then vertically down the side of the LD Devices 18A, 18B, to flow beneath the “In-filler” LD Device containers 18B and then vertically up the side of the LD Devices to then flow over the next LD Device(s) 18A Braced Row.

FIG. 8 Diagram C schematically shows how each panel 161-165 is joined on its long side with the sealed ceiling sheets 20 of adjacent panels clasped between the clamping strip 56 and the base plate 57 that is fixed to the standoff 59 which in turn, is mounted on the floor plate 58. The floor plate 58 is secured using tethers 117 that are welded to the base 160 together with tie down straps. Sand 87 can also be optionally used and layered on the floor 160 to provide ballast and stability against wind forces, to assist in securing the base plate 57 and provide thermal ballast to assist in maintaining a more constant water temperature and controllable algal growth in the multi-panelled sealed tent 12.

The standoff 59 is structured to provide a gas barrier between adjacent panels by hanging a barrier plate 60 from the base plate 57 to below the water level 14A along the length of the panel. This is schematically shown in FIG. 8, Diagram D. In this way if a leak or damage occurred in the ceiling sheet 20, then the panel, say panel 162, in which the leak occurred can be shut down and excluded from the gas regime of the other panels 161, 163,164 and 165 of multi-panelled sealed tent 12. Carbon dioxide/oxygen gas flow between panels and hence the entire multi-panelled sealed tent 12, is enabled through pipes connecting each panel with its adjacent panels, for example panel 162 has adjacent panels 161 and 163. The connecting pipes are valved to moderate gas flow and also enable the connection of downstream panels, isolation of panels and rerouting of gas flow in the case of a leak or damage to a panel's seal or ceiling sheet 20.

The barrier plate 60 between panels also hinders the reverse dilution of oxygen gas back through the panels towards the “Near End” (i.e. in the above example back from 165 towards 161) assisting in producing an ever increasing proportion of oxygen in the stored gas CO₂ and Oxygen 32 as the gas progresses from panel to panel of the multi-panelled sealed tent 12, as directed towards the “Far End” by the gas reticulation used to connect adjacent panels. Thus, the “Near End” gas 32 has a high proportion of CO₂, whereas the “Far End” gas 32 has a high proportion of oxygen, which then can be fed to the closed system combustion stage 38.

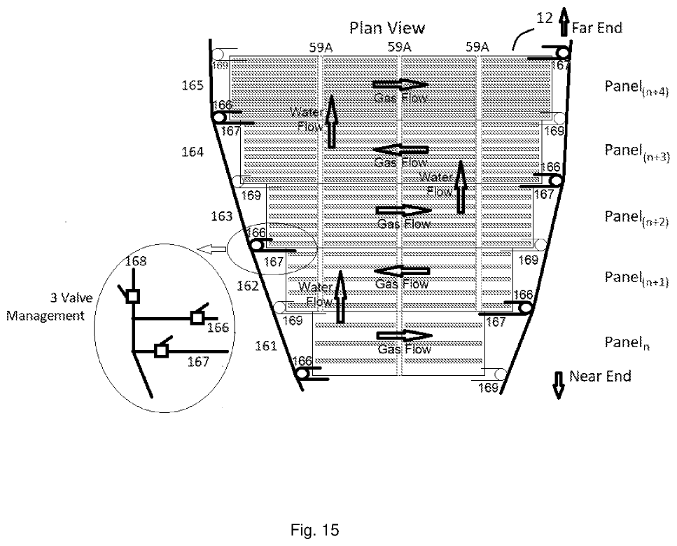

Gas reticulation or circulation within the multi-panelled sealed tent 12 is diagrammatically presented in FIG. 15 showing a subset (161 to 165) of all panels, wherein CO₂ gas is injected 166 into a panel preferably as far as possible from the gas outlet pipe 167 of that panel. The capture of oxygen is effected in each panel (say for example panel 161) and therefore the gas outlet pipe 167 will contain that proportion of oxygen generated and other gases contained within that panel 161 and which is then injected 166 into the next panel (say for example panel 162). Thus the proportion of oxygen steadily increases as the gas 32 passes from one panel to the next (Refer to FIG. 4), until the Oxygen:CO₂ gas mixture is captured at the Far End gas offtake 36.

The Valves on Gas Outlet 167 from a panel, the Gas Inlet 166 into the next panel and the Gas Bypass 168 to direct gas past a panel, are preferably configured to establish a gas flow that routes the gas over the entire length of each panel with the alternate valve sets at 169 closed to enforce that flow as presented in FIG. 15, Valves 166,167,168 and valve sets 169 can be reconfigured for gas flow to bypass a panel to enable maintenance of the multi-panelled sealed tent 12 or to otherwise achieve a desired gas flow pattern. The result is an extended and elongated route for gas flow over the algae growth medium and higher time for carbon dioxide or other gas (e.g. NOx) absorption by the algae growth medium. Conversely, more time is also provided for oxygen produced by respiring algae to diffuse out of the algae growth medium.

### The LD Device 18A, 18B Cleansing Device 96

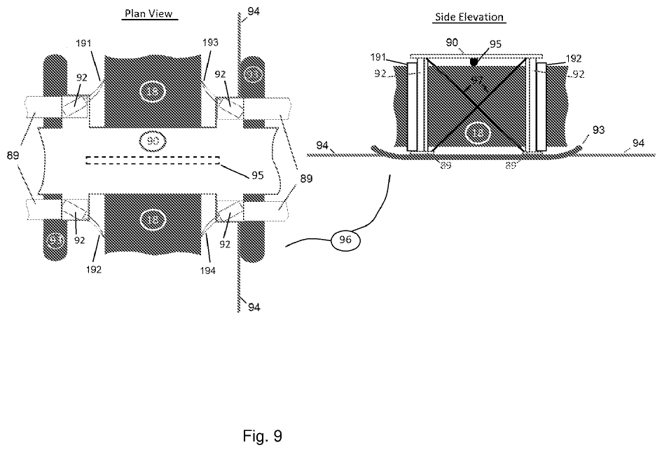

The surfaces of each LD Device 18A, 18B are predisposed to algae buildup. The top and side surfaces of the LD device 18A, 18B are kept clean of algae buildup, by installing a cleansing device 96, as shown in FIG. 9, which straddles the LD Device 18A, 18B and which can be pulled along the LD Device 18A, 18B using a rope and winching mechanism as described below. Alternative moving mechanisms for cleansing device 96 may be employed in alternative embodiments.

Each cleansing device 96 has a minimum of two pairs of flexible blades 191,192 and 193,194 and/or brushes that engage each LD Device 18A, 18B, each flexible blade 191,192 and 193,194 and/or brush preferably standing at least the height of the LD Device 18A, 18B and each blade or brush being fixed to a blade and/or brush holder 92 that forms a structural pillar connecting a pair of skids 93 (using standoffs 89) to a Rigid Top Plate 90 that straddles each LD Device 18A, 18B.

It should be noted that if HDPE (with relative density of 0.96 gm/cc) or other material that has a relative density less than water is used in the construction of the cleansing device 96 then a counterweight may be required on the cleansing device 96 to stop it floating upwards into the LD Devices 18A, 18B.

Brushes, which may comprise a pair of flexible blades 191,192 and 193,194, are configured for bi-directional motion, where at least one of a pair of flexible blades and/or brushes, for example blade 191 of pair 191,192 is effective for at least one direction, and the other flexible blade 192 of the same flexible blade pair 191,192 is effective for the opposite direction.

Each flexible blade of each brush 191,192 and 193,194 is preferably bevelled (slanted) against the side of the LD Device 18A, 18B such that the force of water as the flexible blade and/or brush 191, 192 and 193, 194is pulled along the LD Device 18A, 18B holds the operating blade and/or brush within each pair of flexible blades 191,192 and 193,194 against the side of the LD Device 18A, 18B.

The pair of skids 93 support the cleansing device 96 that service one or more LD Device(s) 18A, 18B. A plurality of cleansing devices 96 is secured to a rope/chord 94 that pulls that plurality of cleansing devices 96, such that the skids 93 ride on the floor 160 (or optional sand ballast 87) of the multi-panelled sealed tent 12.

The pair of skids 93 are rounded at their ends to reduce any snagging against the LD Device 18A, 18B and are curved upwards at each end to reduce any snagging with the floor 160 (or optional sand ballast 87) of the multi-panelled sealed tent 12.

The structural pillars 92 preferably should be braced 97 for additional structural support.

One or more cleansing devices 96 are secured together using a common Rigid Top Plate 90 (as per FIG. 9,12) that straddles LD Devices 18A, 18B (herein generically known as “18”). Also, adjacent cleansing devices 96 can be secured together by sharing common standoffs 89. This allows the plurality of cleansing devices 96 to be connected, top (90) and bottom (89) at appropriate spacings between LD Devices (for each panel) and act as a contiguous unit and share a common rope or chord 94 and winching mechanism 111.

One or more cleansing devices 96 is pulled from one end of the LD Device(s) 18 to the other (and vice versa) using a rope or chord 94.

The cleansing device 96 should preferably have a brush or blade 95 attached to the Rigid Top Plate 90 to clean the top of the LD Device(s) 18A, 18B.

There are as many cleansing devices 96 as there are LD Devices 18A, 18B and in the above example (and not limiting the invention) there are 21 cleansing devices 96 across the Far End panel of the multi-panelled sealed tent 12, connected using one or more Rigid Top Plates 90 and with adjacent cleansing devices 96 connected at the base using common standoffs 89. Each plurality of cleansing devices 96 connected by a common Rigid Top Plate 90 is pulled with a set of ropes/chords 94.

### Pull Ropes/Chords 94 and Winches 111

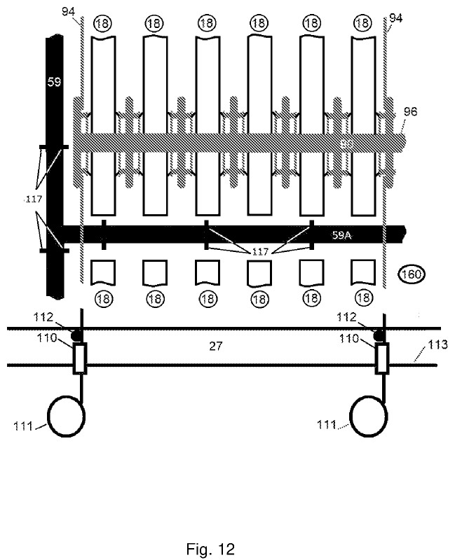

The pull ropes/chords 94 are operated with the aid of winches 111 (FIG. 12). Winches 111 are conveniently driven by electric motors (not shown).

At the bottom edge of the curbing 27, is a water resistant (preferably nylon) pulley(s) 112 secured to the base of the curbing 27 of the “multi-panelled sealed tent” 12 to redirect the rope/chord(s) 94 up the wall of the “multi-panelled sealed tent” 12, to a pipe(s) 110 that sheathes the rope/chord(s) 94 from a point below the liquid algae growth medium water level 14A, to a location beyond the top 113 of the curbing 27 and outside of the multi-panelled sealed tent 12. The sheathing pipe(s) 110 therefore, provide a sealed airtight egress for the rope/chord(s) 94.

On exit from the sheathing pipe(s) 110, the rope/chord(s) 94 is wound on a winch(s) 111 which provides winding tension in the rope/chord(s) 94, when pulling the cleansing device(s) 96 along the LD Device(s) 18A, 18B, towards the pulley 112.

The rope/chord 94 is wound on a winch 111 which provides winding tension in the rope/chord 94, when pulling the plurality of cleansing device(s) 96 along the LD Device(s) 18A, 18B, towards the winch 111 that is winding the rope/chord 94 (meaning that the winch 111 at the other end of the rope/chord 94 is un-winding).

The sheathing pipes 110 that sheathe the rope/chord(s) 94 from outside of the multi-panelled sealed tent 12 to an internal location(s) below the liquid algae growth medium water level 14A of the liquid algae growth medium 14 can also be used as sampling point(s) for measurement of algae density and nutrient if located on the long side of the multi-panelled sealed tent 12 (and used for the purpose of sampling and not winding). Suction of the liquid algae growth medium 14 up the sheathing pipes 110 is the preferable method of sampling.

### Injection of Algae Growth Medium 14 into Multi-Panelled Sealed Tent 12

With reference to FIGS. 4, 10 and 11, liquid algae growth medium 14 promoting algae growth in the multi-panelled sealed tent 12, is injected into the multi-panelled sealed tent 12 via an inlet pipe 24 fixed to the “near end” plate 17. The inlet pipe(s) 24 can be used to
a. recirculate liquid algae growth medium 14 from recirculation pipe 31; and/or
b. replace water consumed by algae and/or lost to the process with top-up water from reservoir(s) 29; and/or
c. inject or otherwise deliver or introduce matter such as nutrients via recirculation pipe 31 and/or reservoir(s) 29; and/or
d. introduce algae seed to promote algal growth via recirculation pipe 31 and/or reservoir(s) 29;
   the source and amount of which, when that source is divisible to one or more reservoir(s) 29, is controlled by a valve(s) 33.

In this embodiment, inlet pipe 24 is operable to inject, or otherwise feed, introduce or deliver, matter into the multi-panelled sealed tent 12 via an injection, or feed/delivery via pipe 30 and/or pipe 31 (which are controlled by a valve(s) 33) to supply the inlet pipe 24.

It may be appreciated that nutrient matter may be injected through the inlet pipe 24, though additional injector(s) may be provided if required. For example, in embodiments, matter such as one or more nutrients suitable for the algae being grown, may be injected. An algal growth medium 14 as known in the art is suitable for provision of such nutrients.

The inlet pipe(s) 24 directs the liquid algae growth medium 14 through the “near end” plate 17 of the multi-panelled sealed tent 12 at an injection rate commensurate with the desired algae density profile and water depth 14A required over the length of the multi-panelled sealed tent 12.

### Injection of Carbon Dioxide into “Multi-Panelled Sealed Tent” 12

Carbon dioxide 44 sourced from the closed system combustion stage 38 and/or carbon dioxide 43 from the closed system fermentation stage 40 is directed via a manifold to multi-panelled sealed tent(s) 12 using valve(s) 34 at or about the “near end” of the multi-panelled sealed tent(s) 12 and via the “near end” plate(s) 17.

The direction of carbon dioxide 43,44 flow from the “near end” plate 17 to the “far end” plate 16 (where it becomes carbon dioxide/oxygen 37), though preferred is not limiting, as there may be occasions in which the direction of carbon dioxide 43,44 flow may, for other design reasons, need to be from “far end” plate 16 to “near end” plate 17.

In this embodiment, gas in the form of carbon dioxide (CO₂) 43,44, is injected 45 to maintain levels of CO₂ in the CO₂ storage facility 32, which forms the means for storing carbon dioxide, here a buffer, to balance the generation of carbon dioxide in closed system combustion stage 38 and closed system fermentation stage 40 with carbon dioxide requirement determined by algal CO₂ uptake rate in the algae multi-panelled sealed tent system 12.

The CO₂ storage facility 32, contains, or otherwise acts as a store or “trap” for CO₂ and contained under a flexible but airtight translucent sheet 20 (refer to FIG. 3,4).

Without wishing to be bound by theory, CO₂ uptake rate of the algae in algae multi-panelled sealed tent system 12 may be less than CO₂ production in the closed system combustion stage 38 and/or closed system fermentation stage 40. In that case, the CO₂ is not wasted or vented, it is stored in storage facilities 32 until algal CO₂ uptake rate requires CO₂ stored in storage facility 32 to feed the waterborne algae.

The above does not prevent inclusion of additional and different CO₂ buffering or storage units within system 100 (for example, pressure vessels). Further, it will be understood that the invention is not limited to use of closed algae growth and oxygen generation stage 10 vessels such as the algae multi-panelled sealed tent system(s) 12 described herein, though the algae multi-panelled sealed tent system(s) 12 are advantageous for the growth of algae and other organisms.

The reticulation of CO₂ gas inside the multi-panelled sealed tent 12 is described above at paragraphs [00175] to [00178].

### “Multi-Panelled Sealed Tent” End Plates 16 & 17

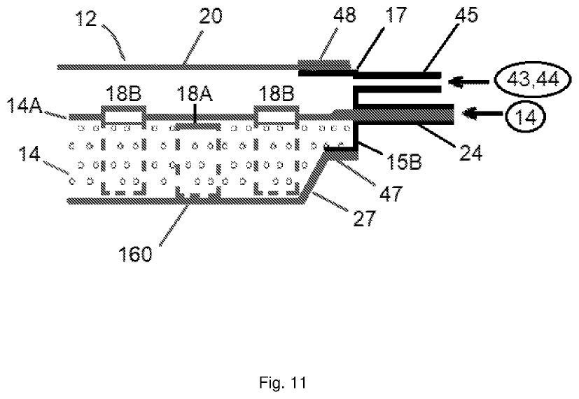

The “near end” plate 17 preferably has a back wall 15B (FIG. 11) of sufficient strength to hold the water pressure of the liquid algae growth medium 14 in the “multi-panelled sealed tent” 12. The back wall 15B can be secured in place either by supporting earthworks or foundational structure to stop the “near end” plate 17 pulling away from the multi-panelled sealed tent 12.

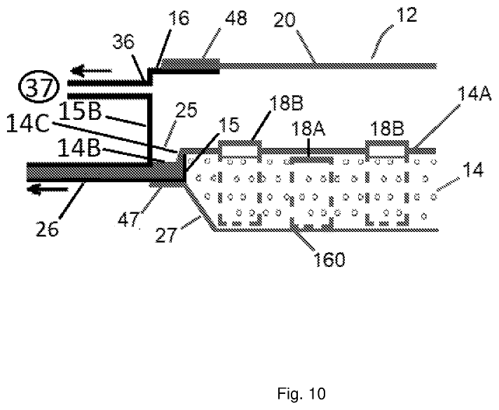

Similarly, the “far end” plate 16 (FIG. 10) preferably has a back wall 15B of sufficient strength to hold the water pressure of the liquid algae growth medium 14 in the multi-panelled sealed tent 12. Back wall 15B should be secured in place either by supporting earthworks or foundational structure to stop the “far end” plate 16 pulling away from the multi-panelled sealed tent 12.

The “far end” plate 16 has a dam wall 15 that can be adjusted in height, to accommodate different sunlight radiation energies that occur across seasons. Adjustment is preferably achieved by fixing a different dimensioned dam wall against a seat in the end plate 16, making it possible to vary the design depth 14A to accommodate different design algae production outcomes. Algae production outcomes will vary by the season. Seasonal variations in incident light angles, light intensities and sunlight duration will, other than effecting algae growth rates, will also effect water temperature necessitating larger or smaller water mass (the design depth 14A) to optimise growth conditions.

The liquid algae growth medium water surface 14A is intended to closely approximate the design depth of “multi-panelled sealed tent(s)” 12.

The dam wall 15, should be sufficiently strong to hold the design depth of multi-panelled sealed tent(s) 12 without bending mid span. Dam wall 15 preferably has a ridge that forms a rim on the top edge to provide structural support against bending, and which can be supported from the back wall 15B, which itself is supported as described above.

The dam wall 15 has a rim below the surface water level 14A of the multi-panelled sealed tent 12. Water in the collection trough 25 of the “far end” plate 16 has a surface water level 14B, which is below or less than the water level 14A of the “multi-panelled sealed tent” 12. Water moving from the multi-panelled sealed tent 12 surface water level 14A to the water level 14B, under the action of pump 22, results in a waterfall 14C.

Liquid algae growth medium 14 is delivered via outlet pipe 26 to the “algae harvesting and downstream processing” stage 70.

The pump 22 is conveniently a water pump which is operable to pump or otherwise remove liquid algae growth medium 14 from the “far end” plate 16 via the outlet pipe 26 and direct it to the “algae harvesting and downstream processing” stage 70.

### Optional Use of Solar Distillation Trays to Concentrate Algae Medium

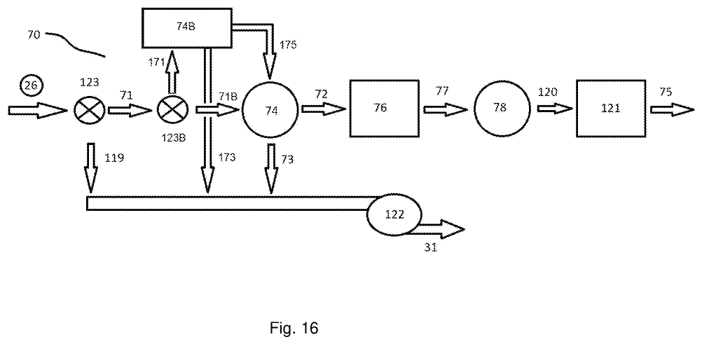

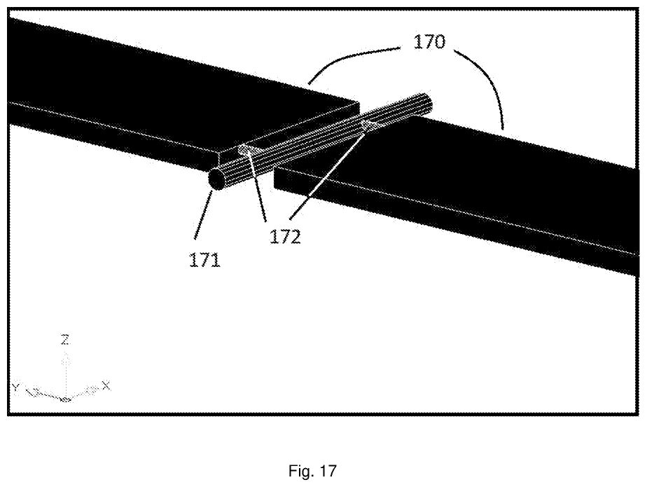

An optional solar distillation and algae flocculation system 74B, can be inserted in the flow of liquid algae growth medium 71 prior to the concentration of the algae medium 71 by the centrifuge(s) 74 for downstream biofuel processing 74,76,78,121 (FIG. 16). By using distillation trays 170 (FIG. 17) that allow approximately 4 cm depth of medium to be exposed to sunlight over the length of each distillation tray 170. The distillation trays 170 are preferably arranged in pairs to share the same algae medium feeder pipe 171, from which inlet injectors 172 are connected to supply the trays 170 with liquid algae medium.

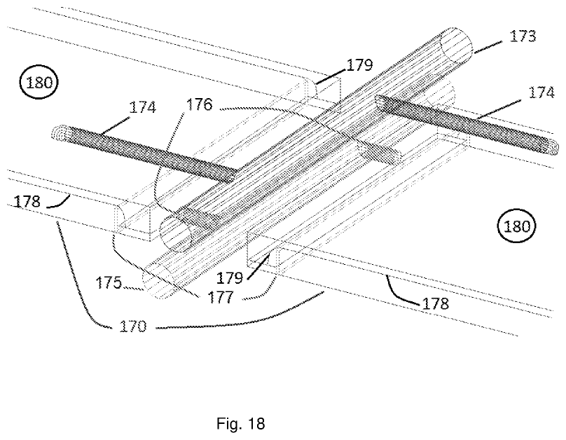

Adjacent distillation trays 170 are connected in like manner as above to optimize the extraction of water vapour and concentrated algae medium 180 as shown in the wireframe diagram of FIG. 18. Water vapour/steam is optionally extracted using vents/extraction pipes 174 which feed the water vapour extraction manifold 173. To assist the process, the extraction of water vapour/steam can be done under negative pressure.

The concentrate algae medium water level 178 is maintained by a weir 177 over which flows by way of a waterfall 179 the concentrated algae medium 180 which is extracted by use of extraction pipe(s) 176 which in turn feeds the Algae Medium concentrate extraction manifold 175.

The Algae Medium concentrate extraction manifold 175 and the optional water vapour extraction manifold 173 service the distillation trays 170 laid side by side, in a similar manner as the algae medium feeder pipe 171 feed the trays at the alternate tray ends.

Because of the temperatures involved in the solar distillation process within the tray, it is preferable to use a rolled steel tray with a glass or acrylic ceiling to construct the distillation trays 170.

Without wishing to be bound by theory, calculations show that a collection of distillation trays 170 similar in the sum of area to that of the multi-panelled sealed tent 12 is sufficient to boil-off approximately 50% of the water in the medium, and at the same time lyse the algae cells (through the boiling action) to separate lipids for biofuel processing. End caps at each end of the distillation tray 170 can be constructed to create a concentrated algae medium waterfall 179 and extraction at one end, and the medium supply 172 at the other end. This optional system reduces the amount of electrical energy required to drive the centrifuges 74 that concentrate the algae medium 71, thereby reducing internal system 100 energy requirements and maximizing the available biofuel for market from the system.

### Concentration of Algal Medium

As alluded to above, algal growth in closed algae growth and oxygen generation stage 10 and its algae multi-panelled sealed tent system(s) 12 is aimed, in preferred embodiments at the production of biofuel 75. Biofuel can be Biodiesel (a fatty acid methyl [or ethyl] ester), Renewable Diesel (a paraffin and/or isomers of paraffin) and/or other paraffinic fuels such as Sustainable Aviation Fuel. In this embodiment, the target algal concentration is 0.16% by volume in the liquid algae growth medium 14 at harvesting or collection for biofuel production from the “far end” plate 16 via outlet pipe 26. Further concentration of algae is needed before processing into biofuel in the biofuel production stage 70. It will be understood that the 0.16% by volume algae target is exemplary and not intended to be limiting.

A portion 119 (in FIG. 16) of the liquid algae growth medium 14 is diverted at valve 123 to be supplied to the “near end” plate 17 (via pump 122 and pipe 31) as seed for further algae growth.

The remaining liquid algae growth medium 71 at the harvested algae concentration is divided into two streams at valve 123B, one stream 71B is directed to the centrifuge(s) 74, and the other stream 171 is directed to an optional algae growth medium 171 distillation tray and algae flocculation system 74B (which may include a flocculation tank) which produces algae concentrate and directs it 175 into the centrifuge 74. Algae flocculation, as known in the art, requires either low or high pH levels where a high pH of 9.5 (or above)—which pH promotes flocculation—is obtained from using substances such as for example, ammonia (which can then be redirected 173 and 73 to the multi-panelled sealed tent system 12 as fertiliser). Algae flocculation produces a conglomerate of algae which can be either precipitated or decanted out of solution and delivered 175 to the centrifuge 74. The depopulated solution containing high concentrations of ammonia may then be distilled in the distillation trays (with the option of ammonia recovery) to not adversely affect the pH levels of the algae growth medium 14 when the solution is returned 173 to pump 122 as recirculated algae growth medium 14 containing additional nutrients as required. Using the centrifuge 74 and optional distillation tray and algae flocculation system 74B, the combined algae conglomerate is concentrated to preferably 40% to 75% by volume 72, and the reject water 173 from the optional distillation tray and algae flocculation system 74B and the centrifuge 74 process reject water 73 is directed to pump 122 to then be supplied to the “near end” plate 17 via pipe 31 as recirculated algae growth medium 14 containing additional nutrients, as required.

Centrifuge(s) 74 are used for the further algal concentration. Industry available centrifuges such as the Dolphin centrifuge (as described at 74, the contents of which are hereby incorporated herein by reference) rated at 64 litres/min and one which can support multiple algal multi-panelled sealed tents 12 as described in this embodiment. The centrifuged algal conglomerate is concentrated to approximately 50 wt % algal biomass by the centrifuge process.

### Extraction of Lipids and Conversion to BioFuel

The desired algal conglomerate of approximately 50 wt % concentration in the processed medium 72 is then passed through an intense sonication device 76 to lyse the cell walls of the algae. Hexane or other solvents, such as a mixture of hexane and ethanol (for example mixed in a 3:1 volume ratio) are also contacted in the sonication process with the medium 72 to extract the lipids from the lysed algal cells through solvent extraction. The solvent, for example hexane or a hexane: ethanol mixture, can be recovered from the product lipids by distillation.

Industry available ultrasonic devices may conveniently be used for intense sonication. For example, lipid extraction may be conducted using commercially available ultrasonic units, such as two Heilsher 4 kW ultrasonic units (as described at https://www.hielscher.com/algae_extraction_01.htm the contents of which are hereby incorporated herein by reference) which are capable of processing 800 litres of algal conglomerate 72 per hour supporting production from up to 30 “multi-panelled sealed tents” 12 as described above.

The solution 77 resulting from the sonication 76 is passed through another centrifuge 78 similar to the centrifuge 74 described for use in algal concentration. This centrifuge separates water, biomass and the lipid oil-hexane solvent mixture, and is capable of supporting production of up to 60 multi-panelled sealed tents 12 as described above. For purposes of example, the hexane in the lipid oil-hexane solvent mixture is distilled off at 69° C. and with a heat of vapourisation (enthalpy requirement) of approximately 31.5 kJ/mol hexane.

The lipid containing oil 120 is then:
1) optionally processed to biodiesel 75 through conventional processing involving transesterification 121, preferably using ethanol rather than methanol as forming an ethyl ester based biodiesel is expected to result in lower emissions than a methyl ester based biodiesel. Ethanol is also produced by system 100 and methanol handling may raise safety issues; and/or
2) optional conversion of the lipids to a paraffin oil such as Renewable Diesel or Aviation Fuel is conveniently conventional in design and operation utilizing hydrogen under high temperature and pressure over a catalyst to “hydrogen process” the lipids into paraffins using two common process stages called “Hydrotreating” and “Hydrocracking”, in which case process 121 is then a “hydrogen process” and biofuel product 75 is then a paraffinic oil such as Renewable Diesel or Aviation Fuel.

### Sensors

The “closed algae growth and oxygen generation” stage 10 preferably comprises a sensor system including sensor(s) for process control. Individual sensors within the sensor system are conveniently operable to monitor, sense and capture or otherwise gather or measure sensor data and/or information associated with or relating to one or more characteristics, properties and parameters of the “closed algae growth and oxygen generation” stage 10, the surrounding environment, or components, systems or devices associated therewith or coupled thereto. For example, the sensor system is conveniently operable to sense and gather sensor data relating to a state of “closed algae growth and oxygen generation” stage 10 and/or a state of the environment surrounding the closed algae growth and oxygen generation stage 10.

Preferably, the sensor system comprises a depth or level sensor operable to measure the water level 14B at the “far end” plate 16 of the multi-panelled sealed tent 12, to assist the control of inflow of liquid algae growth medium 14 into the multi-panelled sealed tent 12 though inlet 24. Further, at least one pressure sensor is desirably used to measure the pressure of gas in storage facility 32 to protect the translucent seal 20 from over-inflation and also ensure that there is sufficient CO₂ available in the storage facility 32 to support algal growth targets.

Conveniently, the pressure measuring equipment is operably coupled to the injector 45 so that CO₂ 43,44 is injected in the required amounts at the required times.

Other sensors may be included to, for example, monitor electrical generation and equipment, the closed system combustion stage 38 and closed system fermentation stage 10 gas flows and temperatures, irrigation systems, evaporation, distillation and permeation systems, and feedstock flow rates.

### EXAMPLE

A multi-panelled sealed tent 12 as described above is constructed from seven metre wide PE (Polyethylene) base sheets 160 and a polyethylene variant (such as for example Polyethylene Terephthalate) for the top sheet 20, forming panels that range between approximately 44 meters and 100 meters long depending on the panel to be constructed, and each panel broken into approximately 20 m sections. The Near End panel has a sheet length of two 24 m sections to construct a 44 m floor (schematically shown in FIGS. 8 and 15), those sections joined in the middle of the panel using a panel section brace 59A. Because the next and adjacent panel has more LD Devices 18, to maintain an equal volume of medium 14 in the second panel (to that of the first panel) the panel length is longer, until the “Far End” panel has a 100 m length floor, preferably broken into five 20 m sections and joined with panel section braces 59A. A system 100 with 21 panels constructed in this manner and with 1.67 m of water height provides a nominal capacity of approximately 6,000 m³ of water (which excludes the space occupied by the LD Devices 18). The storage facility 32 of the same multi-panelled sealed tent 12 would hold about 9,000 m³ of gas containing CO₂ and oxygen. At 60%:40% ratio of Oxygen:Carbon Dioxide by volume at the “Far End” of the multi-panelled sealed tent 12, this storage space would sustain algal growth in algae multi-panelled sealed tent system 12 for approximately eight days at design production without a CO₂ feed.

Combustion of bagasse 4 from one hectare of sweet sorghum is expected to produce about 26 Tonnes of CO₂ from 1 hectare of 2 crops of sweet sorghum per year. Fermentation of juice from the same one hectare of sweet sorghum would also be expected to produce about 5.2 Tonnes of ethanol/year and about 5 Tonnes of CO₂/yr. In total about 31 Tonnes of CO₂ could be generated per year from one hectare of sweet sorghum. A single multi-panelled sealed tent 12 as described above may be balanced against about eleven hectares of sweet sorghum that produces two crops per year at about 160 Tonnes per year/hectare as described above.

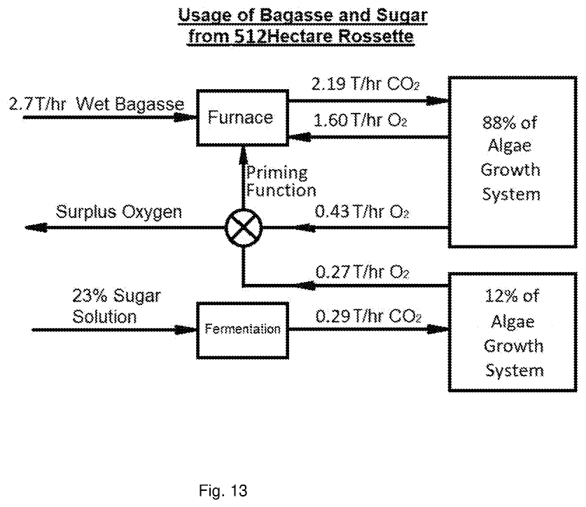

For example, it is estimated that if 512 hectares of sweet sorghum crop and with two harvests per year, is processed by a “proximate crop processing plant” 1, 33,000 Tonnes of juice may be produced per year. This juice, (albeit concentrated as molasses for longevity) when fermented on a daily basis over a year will produce very approximately 0.29 Tonnes CO₂ per hour (as schematically described in FIG. 13). This is predicted to be sufficient to support algal growth of very approximately 12% of a compatible multi-panelled sealed tent(s) 12. CO₂ generated by fermentation is desirably stored 32, on startup, to prime the algae growth and oxygen generation system 10.

Fermentation enables the startup of the algae growth and oxygen generation system 10 whereby the algae multi-panelled sealed tent(s) 12 that is driven by the fermentation system, produce very approximately 0.27 Tonnes/hour of oxygen (averaged over a 24 hr day). The oxygen is stored in the gas storage facility 32 then released as an oxygen/carbon dioxide mix 37 to feed the furnace(s) of the closed system combustion stage 38, which on startup may operate intermittently.

When 0.27 Tonnes/hour of Oxygen (averaged over a 24 hr day) is fed to the furnace(s) of the closed system combustion stage 38, it is expected that 0.37 Tonnes of CO₂/hour is produced, in that about 1.38 times more CO₂ is produced than oxygen consumed (by weight). Thus, there is an exponential increase in ability to support additional algae growth in multi-panelled sealed tent(s) 12 until all the bagasse 4 that is apportioned over a year is consumed at an hourly rate by the “closed system furnace” 38 supporting the multi-panelled sealed tent(s) 12 that are dimensioned against the example 512 Hectares of crop and leaving an abundance of oxygen as surplus.

It will be appreciated by those skilled in the art that variations and modifications to the systems and methods for generating and using carbon dioxide described herein will be apparent without departing from the spirit and scope thereof. The variations and modifications as would be apparent to persons skilled in the art are deemed to fall within the broad scope and ambit of the invention as herein set forth.

1-51. (canceled)

52. A system for generating and using carbon dioxide, the system comprising:
(a) a closed system combustion stage for combusting a fuel and producing an off gas containing carbon dioxide and a nitrogen containing gas including NOx; and
(b) an algae growth and oxygen generation stage for receiving said off gas generated in the closed system combustion stage wherein said algae growth and oxygen generation stage through the action of algae:
(i) converts said carbon dioxide to a chemical product, and oxygen; and
(ii) provides a gas stream to the closed system combustion stage, said gas stream substantially comprising a mixture of algal produced oxygen in major proportion and carbon dioxide and nitrogen containing gas including NOx from said off gas in minor proportion
wherein the algae growth and oxygen generation stage comprises at least one sealed tent containing a growth medium for the algae, said at least one sealed tent providing an extended and elongated route for circulation of gas over algae growth medium in said at least one sealed tent; and
wherein said at least one sealed tent communicates with the closed system combustion stage for delivery of off gas containing carbon dioxide to the at least one sealed tent and further communicates with the closed system combustion stage for providing said gas stream substantially comprising a mixture of algal produced oxygen in major proportion and carbon dioxide and nitrogen containing gas including NOx in minor proportion to said closed system combustion stage.

53. The system of claim 52, comprising a plurality of sealed tents.

54. The system of claim 52, wherein said at least one sealed tent is a multi-panelled sealed tent.

55. The system of claim 52, wherein the off gas contains carbon dioxide, NOx and components selected from the group consisting of carbon monoxide, SOx, minerals and acid gases; and wherein said closed algae growth and oxygen generation stage contains a gas comprising a mixture of carbon dioxide and oxygen, said closed algae growth and oxygen generation stage:
(i) consuming at least a portion of the off gas components as a nutrient;
(ii) converting said carbon dioxide to a chemical product, and oxygen;
(i) storing a gas mixture substantially comprising carbon dioxide and oxygen; and
(ii) providing a portion of said gas mixture from (iii) to the said closed system combustion stage.

56. The system of claim 55, wherein a proportion of said gas from said closed system combustion stage being NOx gases is utilized as nitrogen fertiliser in the closed algae growth and oxygen generation stage.

57. The system of claim 55, wherein said chemical product is further processed into a biofuel.

58. The system of claim 52, wherein said gas containing a mixture of oxygen in major proportion and carbon dioxide in minor proportion is contained within the closed algae growth and oxygen generation stage so as to be used for continuous combustion or to enable intermittent combustion in the closed system combustion stage.

59. The system of claim 58, wherein carbon dioxide in the gas mixture is at least partially produced during combustion and oxygen in the mixture is at least partially produced by the closed algae growth and oxygen generation stage.

60. The system of claim 52, including a carbon dioxide balancing system for balancing carbon dioxide generation from upstream carbon dioxide generation stages with carbon dioxide requirements in the process for organism growth to produce biomass, said carbon dioxide balancing system including a carbon dioxide storage means for storing carbon dioxide generated by the closed system combustion stage in excess of the required carbon dioxide uptake rate of the carbon dioxide respiring algae.

61. The system of claim 53, wherein said at least one sealed tent comprises: a liquid algae growth medium bearing algae flowing from one end of the sealed tent to the other end of the sealed tent, thence into a collector for harvesting.

62. The system of claim 61, wherein said at least one sealed tent comprises a light diffusion (LD) subsystem to expose organisms at depth to light energy.

63. The system of claim 52, wherein said sealed tent has a supply of seed algae at one end.

64. The system of claim 63, wherein circulation of water through the sealed tent provides a progression of algae density from seed density at the one end of the sealed tent to harvestable density at the other end.

65. The system of claim 64, wherein recirculated water from harvested algae from one end of the sealed tent contains a portion of the harvested algae as seed algae delivered to the other end of the sealed tent.

66. The system of claim 62, wherein the light diffusion subsystem comprises one or more sealed transparent and impervious (to water and air) light diffusion (LD) device(s) elongated within the panels of a multi-panelled sealed tent.

67. The system of claim 66, wherein parallel aligned light diffusion (LD) devices are secured in each panel in a manner that forces the flow of liquid algae growth medium across the top of one light diffusion (LD) device and below an adjacent light diffusion (LD) device.

68. The system of claim 67, wherein light diffusion (LD) devices in the form of adjacent and parallel sealed containers are constructed to secure, optionally by tethers or braces, one sealed container to the floor of the multi-panelled sealed tent and submerged below the water level of the liquid algae growth medium and the adjacent sealed container is floated above the surface of the liquid algae growth medium.

69. The system of claim 68, wherein each light diffusion (LD) device that is floated above the surface of the liquid algae growth medium is likewise floated a sufficient distance from the floor to allow the movement of water underneath the light diffusion (LD) device.

70. The system of claim 69, wherein each light diffusion (LD) device that is floated above the surface of the liquid algae growth medium is supported vertically with sufficient buoyancy to hold it upright when the base is tethered either to the floor of the at least one sealed tent, or alternatively braced to adjacent light diffusion (LD) devices that are themselves secured to the floor of the at least one sealed tent.

71. The system of claim 70, wherein the movement of liquid algae growth medium is sequentially directed over the light diffusion (LD) device that is submerged and secured to the floor of the at least one sealed tent, vertically down the sides of the adjacent light diffusion (LD) devices, to flow beneath the buoyed (floated) LD device and then vertically up the sides of the next adjacent LD devices to then flow over the next submerged LD device.

72. The system of claim 66, wherein width of a light diffusion (LD) device about the surface of the liquid algae growth medium is of sufficient width to capture light that the average Photosynthetically Active Radiation (PAR) emitted through the sides of the light diffusion (LD) device is at an average sufficient light intensity suitable for growing algae.

73. The system of claim 72, wherein the spacing between each light diffusion (LD) device is commensurate with variable algae density along the “multi-panelled sealed tent” as defined by a determined profile of algae density, resulting from a seed concentration at a “near end” of the multi-panelled sealed tent and a harvestable concentration at the “far end” of the multi-panelled sealed tent.

74. The system of claim 73, wherein the spacing between each light diffusion (LD) device is determined by the maximum light pathway distance from the side of the light diffusion device into the liquid algae growth medium for the determined algae density at that proximate area along the multi-panelled sealed tent.

75. The system of claim 55, comprising an algae separation system to separate algae from water, optionally when the algae form a predetermined volume in water at the end of the closed sealed tent(s) at which the algae is harvested.

76. The system of claim 53, wherein increasing or reducing the number of operational sealed tents operational in the closed algae growth and oxygen generation stage controls oxygen concentration in the gas stream directed to the closed system combustion stage.
